(19) United States
(12) Patent Application Publication
(10) Pub. No.: US 2025/0306036 A1
(43) Pub. Date: Oct. 2, 2025

(54) BIOMARKER COMPOSITION FOR EARLY DIAGNOSIS OF KIDNEY DISEASES, AND METHOD FOR PROVIDING INFORMATION REQUIRED FOR EARLY DIAGNOSIS OF KIDNEY DISEASES BY USING SAME

(71) Applicant: KONKUK UNIVERSITY INDUSTRIAL COOPERATION CORP, Seoul (KR)

(72) Inventors: Sun Hee DO, Hanam-si Gyeonggi-do (KR); Hyosung KIM, Seoul (KR)

(73) Assignee: KONKUK UNIVERSITY INDUSTRIAL COOPERATION CORP, Seoul (KR)

(21) Appl. No.: 19/183,110

(22) Filed: Apr. 18, 2025

Related U.S. Application Data
(63) Continuation of application No. PCT/KR2023/016383, filed on Oct. 20, 2023.

(30) Foreign Application Priority Data
Oct. 20, 2022 (KR) 10-2022-0135454
Oct. 19, 2023 (KR) 10-2023-0140462

(51) Int. Cl. G01N 33/68; G01N 33/573; G01N 33/62; G01N 33/70; G01N 33/84

(52) U.S. Cl. CPC G01N 33/6893; G01N 33/573; G01N 33/62; G01N 33/70; G01N 33/84; G01N 2333/46; G01N 2333/928; G01N 2800/347; G01N 2800/50; G01N 2800/56

(57) ABSTRACT

The present invention relates to a biomarker for early diagnosis of kidney disease and use thereof, and according to a composition, a kit, and a method of an aspect, kidney disease or risk of kidney disease can be diagnosed early with greater accuracy, sensitivity, and specificity. In particular, according to the present invention, based on the International Renal Interest Society (IRIS) guidelines for staging chronic kidney disease (CKD), it is possible to distinguish between a normal group and a risk group (a stage with risk factors), and Stage 1 CKD (IRIS stage 1) or Stages 2 to 4 CKD (IRIS stages 2 to 4), with a sensitivity of 90% or higher and a specificity of 95% or higher.

### CROSS-REFERENCE TO RELATED APPLICATIONS

This application is a continuation of International Application No. PCT/KR2023/016383 filed on Oct. 20, 2023, which claims priority to Korean Patent Application No. 10-2022-0135454 filed on Oct. 20, 2022 and Korean Patent Application No. 10-2023-0140462 filed on Oct. 19, 2023, the entire contents of which are herein incorporated by reference.

### TECHNICAL FIELD

The present invention relates to a biomarker composition for early diagnosis of kidney disease and a method of providing information necessary for early diagnosis of kidney disease using the same.

### BACKGROUND ART

The kidneys are two organs located on both sides of the spine in the lower back region of the human body, and have, in addition to the main function of filtering metabolites and wastes in the body and excreting the filtrates through the urine, a homeostasis maintaining function, which is to keep the body fluid and electrolytes, acidity, and the like within a narrow range, and an endocrine function, which is to produce and activate a number of hormones that are important for maintaining blood pressure, correcting anemia, and metabolizing calcium and phosphorus.

Kidney diseases that cause weakening of such kidney functions include glomerulonephritis, chronic renal failure, acute renal failure, nephrotic syndrome, pyelonephritis, kidney stones, kidney cancer, and the like. Depending on how quickly the deterioration of kidney functions progresses, kidney diseases can be broadly divided into acute kidney injury (AKI) and chronic kidney disease (CKD).

In CKD, kidney functions slowly decline over many months, are usually irreparable and progressive, and progresses to end-stage kidney disease, a condition that often requires dialysis or kidney transplantation. AKI, on the other hand, refers to a rapid deterioration in kidney functions within days or weeks, with common causes including dehydration or low blood pressure, nephrotoxic substances or medications, urinary tract obstruction, and the like. Conservative treatment is usually aimed at ameliorating dehydration through fluid replenishment or removing the cause of kidney strain, to restore normal kidney functions, but depending on the severity of underlying conditions, some cases may progress to chronic renal failure.

Despite the advances in modern medicine, many patients admitted to hospitals suffer from a decline in kidney functions, and in particular, patients with severe disease often require renal replacement therapy due to declining kidney functions. The prevalence of AKI has been reported to range from about 5% of hospitalized patients to about 30 to 50% of patients admitted to intensive care units, and this prevalence continues to increase despite the development of new therapies (Lameire et al., Lancet, 2005; Devarajan, Contrib Nephrol, 2007).

The high mortality rate of AKI can be attributed to a number of factors, but the lack of early diagnosis methods for AKI, which results in the lack of timely treatment, may be a major contributing factor. Traditional methods of evaluating kidney functions may be to measure serum creatinine, which indirectly reflects the degree of kidney functions. However, serum creatinine is affected by subject's weight, age, gender, muscle mass, protein intake, medications, and the like, and thus has the disadvantage of not reflecting changes in kidney functions in real time. In other words, serum creatinine is limited in its ability to diagnose AKI early, as it requires a 50% or more decline in kidney functions to cause elevation of serum creatinine (Belcher et al., Am J Kidney Dis, 2011; Endre and Westhuyzen, Nephrology, 2008). Fortunately, as recent innovations such as functional genomics and proteomics have been developed and applied, various proteins and gene products have been proposed as biomarkers, but there are still limitations in terms of clinical efficacy.

Neutrophil gelatinase-associated lipocalin (NGAL) is a 25 kDa glycoprotein bound to neutrophils or the epithelium of renal tubules, and is a biomarker that is rapidly increased in AKI from a variety of causes. Currently, it is mainly used to diagnose kidney dysfunctions and to determine the prognosis of kidney transplant patients.

In addition, kidney injury molecule-1 (KIM-1) is a protein that is not expressed in normal kidneys, but is strongly expressed starting several hours after kidney injury in the renal tubules of patients with ischemic-reperfusion injury, nephrotoxic drugs, and kidney disease. KIM-1 consists of a cytoplasmic domain and an ectodomain, the latter of which is excreted through the urine and has been studied a lot as a diagnostic biomarker for kidney disease.

However, conventional clinicopathological diagnostic techniques for kidney disease using not only NGAL and/or KIM-1, but also serum creatinine and symmetric dimethylarginine (SDMA) have not been able to distinguish between a normal group and a risk group of kidney disease.

In this regard, the inventors of the present invention have made efforts to develop an optimal index for early diagnosis of kidney disease groups, and in particular, to differentiate between a normal group and a risk group of kidney disease. After measuring the concentration of each of NGAL and KIM-1 in a body fluid sample of a subject, the extent to which each variable, including the concentration of each of NGAL and KIM-1, affects the prevalence of disease was determined by crude logistic regression analysis, and only the variables that are significant at a significance level of 0.05 are combined to determine the functional relation between the variables by multiple logistic regression analysis. Based on the value estimated therefrom, an optimal model (function) with the highest pseudo R2 (explanatory power, the strength of the relation between the dependent and independent variables) is derived. When the derived optimal model is used, the accuracy, sensitivity, and specificity of early diagnosis of kidney disease are found to be significantly high, thereby completing the present invention.

### PRIOR ART DOCUMENTS

### Patent Document

(Patent Document 1) KR 10-2013-0089474 A
(Patent Document 2) KR 10-1657881 B1

### SUMMARY

### Technical Problem

One aspect is to provide a composition for diagnosing kidney disease, including an agent capable of measuring an expression level of a neutrophil gelatinase-associated lipocalin (NGAL) protein, a kidney injury molecule-1 (KIM-1) protein, or a combination thereof, or a gene encoding the same.

Another aspect is to provide a kit for diagnosing kidney disease, including the composition.

Another aspect is to provide a method of providing information for diagnosis of kidney disease, including: measuring an expression level of an NGAL protein, a KIM-1 protein, or a combination thereof, or a gene encoding the same, in a biological sample obtained from a subject; and comparing the measured expression level with expression levels of proteins or a combination thereof, or a gene encoding the same, in a normal group.

Another aspect is to provide a method of treating kidney disease, including measuring an expression level of an NGAL protein, a KIM-1 protein, or a combination thereof, or a gene encoding the same, in a biological sample obtained from a subject.

Another aspect is to provide a method of diagnosing kidney disease, including: measuring an expression level of an NGAL protein, a KIM-1 protein, or a combination thereof, or a gene encoding the same, in a biological sample obtained from a subject; and comparing the measured expression level with expression levels of proteins or a combination thereof, or a gene encoding the same, in a normal group.

Another aspect is to provide a system for diagnosing kidney disease, including: an input unit configured to input a concentration of at least one marker selected from NGAL KIM-1 as measured from a body fluid sample of a subject, or to input, together with the concentration of the at least one marker, a concentration of at least one marker selected from symmetric dimethylarginine (SDMA), creatinine, inorganic phosphorus, amylase, and BUN; a variable setting unit configured to set, as a single or multiple independent variable, the input concentration of the at least one marker, and to set, as a dependent variable, the onset of a CKD risk group (a stage with risk factors), Stage 1 CKD (IRIS stage 1), or Stages 2 to 4 CKD (IRIS stages 2 to 4) based on the IRIS guidelines for staging CKD; an inference engine unit configured to model the relation between the multiple independent variable and the dependent variable by logistic regression analysis to deduce a model equation; and a diagnosis unit configured to determine a subject to belong to a risk group of kidney disease or to be at Stage 1 CKD (IRIS stage 1) or Stages 2 to 4 CKD (IRIS stages 2 to 4), when a value deduced by the model equation is greater than or equal to a predetermined cutoff value, wherein the value is deduced by substituting data for the at least one marker input by the input unit into the independent variable of the inferred model equation.

Another aspect is to provide a computer-readable recording medium having recorded thereon a computer program for executing the method on a computer.

### Solution to Problem

One aspect provides a composition for diagnosing kidney disease, the composition including an agent capable of measuring an expression level of a neutrophil gelatinase-associated lipocalin (NGAL) protein, a kidney injury molecule-1 (KIM-1) protein, or a combination thereof, or a gene encoding the same.

The NGAL is a 25 kDa glycoprotein bound to neutrophils or the epithelium of renal tubules, and is known to play an important role in assessing kidney health or kidney injury. The NGAL may be expressed in response to damage to the proximal tubules or damage to nephrons in the kidney.

The KIM-1 is a protein that is not expressed in normal kidneys, but is strongly expressed several hours after kidney damage in the renal tubules of patients with ischemic and reperfused renal injury, nephrotoxic drugs, and kidney disease. It is one of the proteins that indicates kidney damage and is mainly used as a biomarker mainly for diagnosis or monitoring of acute kidney injury. The KIM-1 consists of a cytoplasmic domain and an ectodomain, and the latter of which is known to be excreted in the urine. The KIM-1 may be increased in expression upon damage to the proximal tubule.

In the present specification, the term “marker” or “biomarker” refers to a substance capable of diagnosing and distinguishing between a normal subject and a subject having a disease, and may include all organic biomolecules, such as polypeptides, proteins, nucleic acids, genes, lipids, glycolipids, glycoproteins, sugars, etc., which show an increase in subjects having a kidney-related disease of the present invention.

In an embodiment, the NGAL or KIM-1 may be used as a biomarker for early diagnosis of kidney disease.

The composition may further include an agent for measuring an expression level of at least one protein or a gene encoding the same, selected from the group consisting of SDMA, BUN, creatinine, inorganic phosphorus, amylase, inulin, and cystatin C.

The SDMA, BUN, creatinine, inulin, and cystatin C may be commercially used as biomarkers for evaluating glomerular filtration rates.

In the present specification, the term “glomerular filtration rate (GFR)” is an index of kidney function and refers to the rate at which the kidneys filter certain substances from the blood. The GFR may indicate the ability of the kidneys to filter waste and substances in the blood and excrete them through urine.

The agent for measuring the expression level of the protein may be selected from the group consisting of a monoclonal antibody, a polyclonal antibody, a chimeric antibody, a ligand, a peptide nucleic acid (PNA), an aptamer, and a nanoparticle that bind specifically to the protein, but is not limited thereto.

Methods for measuring the expression level of the protein may include protein chip analysis, immunoassay, ligand binding assay, matrix desorption/ionization time of flight mass spectrometry (MALDI-TOF) analysis, surface enhanced laser desorption/ionization time of flight mass spectrometry (SELDI-TOF) analysis, radioimmunoassay, radioimmunodiffusion, orchite immunodiffusion, rocket immunoelectrophoresis, tissue immunostaining, complement fixation assay, two-dimensional electrophoresis analysis, liquid chromatography-mass spectrometry (LC-MS), liquid chromatography-mass spectrometry/mass spectrometry (LC-MS/MS), western blot, and enzyme linked immunosorbent assay (ELISA), but are not limited thereto. Therefore, the agent for measuring the protein level may include an antibody that binds specifically to the NGAL protein or the KIM-1 protein.

In the present specification, the term “antibody” may refer to a specific protein molecule directed against an antigenic site. For the purposes of the present invention, the antibody refers to an antibody that binds specifically to the NGAL protein or KIM-1 protein, and may include all of a polyclonal antibody, a monoclonal antibody, and a recombinant antibody. Antibodies may be easily produced using techniques widely known in the art. In addition, the antibody of the present invention includes not only a complete form having two full-length light chains and two full-length heavy chains, but also a functional fragment of an antibody molecule. The functional fragment of an antibody molecule refers to a fragment having at least an antigen-binding function, and examples thereof may include Fab, F(ab′), F(ab′)₂, Fv, and the like.

The agent for measuring the expression level of the gene encoding the protein may be selected from the group consisting of a primer pair, a probe, and an antisense nucleotide that bind specifically to the gene, but is not limited thereto.

In the present specification, the term “primer pair” includes any combination of primer pairs consisting of forward and reverse primers that recognize a target gene sequence, and more specifically, may refer to a primer pair that provides an analysis result with specificity and sensitivity. The nucleic acid sequence of the primer is a sequence that does not match a non-target sequence present in a sample, and thus high specificity may be achieved when the primer amplifies only the target gene sequence including a complementary primer-binding site and does not cause non-specific amplification.

In the present specification, the term “probe” refers to a substance that is capable of binding specifically to a target substance to be detected in a sample, and may include a substance that can specifically confirm the presence of the target substance in the sample through binding. Types of a probe molecule are not limited to those conventionally used in the art, but may preferably be a PNA, a locked nucleic acid (LNA), a peptide, a polypeptide, a protein, RNA or DNA. More specifically, the probe may include a biomaterial derived from or similar to a living organism or manufactured in vitro, and may be, for example, an enzyme, a protein, an antibody, a microorganism, an animal or plant cell and organ, a nerve cell, DNA, and RNA, wherein DNA includes cDNA, genomic DNA, and oligonucleotides, and RNA includes genomic RNA, mRNA, and oligonucleotides, and examples of the protein may include an antibody, an antigen, an enzyme, a peptide, and the like.

In the present specification, the term “antisense oligonucleotide” refers to DNA or RNA, or a derivative thereof, containing a nucleic acid sequence complementary to a sequence in particular mRNA, which binds to the complementary sequence in the mRNA and inhibits the translation of the mRNA into a protein. The sequence of the antisense oligonucleotide may refer to a DNA or RNA sequence that is complementary to the mRNA of the gene and is capable of binding to the mRNA. The antisense oligonucleotide may inhibit the translation, translocation into the cytoplasm, maturation or any other essential activities of the gene's mRNA for its overall biological function. The antisense oligonucleotide may be 6 to 100 bases in length, preferably 8 to 60 bases in length, more preferably 10 to 40 bases in length. The antisense oligonucleotide may be synthesized in vitro by conventional methods and administered into a living body, or the antisense oligonucleotide may be synthesized in vivo. One example of synthesizing the antisense oligonucleotides in vitro may include use of RNA polymerase I. One example of how to synthesize antisense RNA in vivo may include use of a vector with the origin of the multiple cloning site (MCS) in the opposite direction to ensure that antisense RNA is transcribed. Preferably, the antisense RNA may have a translation stop codon within the sequence to prevent it from being translated into a peptide sequence.

In an embodiment, the kidney disease may be acute kidney injury (AKI) or chronic kidney disease (CKD).

The AKI is not particularly limited, but may be any one selected from the group consisting of acute renal failure, acute tubular necrosis, acute tubulointerstitial nephropathy, ischemic AKI, acute pyelonephritis, acute progressive nephritis, and toxic AKI, but is not limited thereto.

The CKD is not particularly limited, but may be any one selected from the group consisting of nephritic syndrome, tubular disorder, renal hypertension, uremia, chronic glomerulonephritis, renal failure and chronic renal failure, but is not limited thereto.

The kidney disease may include one or more diseases selected from the group consisting of diabetic nephropathy, hypertensive nephropathy, glomerulonephritis, polycystic kidney disease, urinary tract obstruction, renal fibrosis, nephritis, pyelitis, renal cancer, hydronephrosis, hemorrhagic fever with renal syndrome, renal tuberculosis, microscopic glomerulosclerosis, diabetic nephropathy, membranous nephropathy, membranoproliferative glomerulonephritis, and nephrosclerotic syndrome, but is not limited thereto.

In an embodiment, the expression level of the protein or the gene encoding the same may be measured in a body fluid sample of a subject.

In an embodiment, the subject may be a mammal, such as a human, a dog, a cat, a cow, a horse, a pig, sheep or a goat, but is not limited thereto.

The subject may be an animal except for a human.

The subject may be selected for early diagnosis of kidney disease based on the presence of one or more pre-existing risk factors selected from prerenal kidney injury, intrinsic renal injury, and postrenal kidney injury.

In the present specification, the term “prerenal kidney injury” refers to kidney injury caused by factors other than extrarenal factors. It usually occurs when there is a problem with blood circulation, which may refer that the kidneys are not supplied with enough blood, resulting in a decline in kidney functions. The prerenal kidney injury may be usually caused by a drop in blood pressure, a decrease in blood volume, and changes in blood viscosity.

In the present specification, the term “intrinsic kidney injury” may refer to kidney injury caused by a problem in the kidney itself, which may be damage to the kidney tissue itself, resulting in a decline in the functions. The intrinsic kidney injury may be caused by cellular damage, inflammation, exposure to toxic substances, infection, hematologic abnormalities, etc. that directly affect the kidney tissue.

In the present specification, the term “postrenal kidney injury” may refer to kidney damage caused by a problem in a renal excretory system, which may be caused primarily by an abnormal inability of the kidneys to drain and accumulation of urine produced by the kidneys. The postrenal kidney injury may be caused by urine not being able to drain normally due to urinary tract infection, ureteral obstruction, bladder obstruction, enlarged prostate, or the like.

The risk factors may include: one or more pre-existing diagnoses selected from congestive heart failure, pre-eclampsia, convulsion, diabetes mellitus, hypertension, coronary artery disease, proteinuria, renal insufficiency, glomerular filtration below normal range, serum creatinine above average range, sepsis, injury to kidney functions, decreased kidney functions, and acute renal failure (ARF); history of one or more surgeries selected from major vascular surgery, coronary artery bypass grafting, and cardiac surgery; or exposure to a non-steroidal anti-inflammatory drug, cyclosporine, tacrolimus, aminoglycoside, foscarnet, ethylene glycol, hemoglobin, myoglobin, ifosfamide, heavy metals, methotrexate, a radiopaque contrast agent, or streptozotocin, but are not limited thereto.

In an embodiment, the subject may not be undergoing renal replacement therapy, but is not limited thereto.

In an embodiment, the body fluid sample may be urine or blood, preferably blood, and more preferably, a plasma or serum sample, but is not limited thereto.

The composition may distinguish between the risk group (a stage with risk factors) and each stage, based on the International Renal Interest Society (IRIS) guidelines for staging CKD.

In the present specification, the term “IRIS guidelines for staging CKD” or “IRIS CKD stages” may refer to the classification criteria of kidney disease as set forth by the International Society of Veterinary Nephrology.

In the present specification, the term “early diagnosis of kidney disease” is meant to include determination of a risk group of kidney disease (a stage with risk factors), Stage 1 CKD (IRIS stage 1), or Stages 2 to 4 CKD (IRIS stages 2 to 4), or early diagnosis of kidney injury or kidney disease.

In the present specification, the term “risk group of kidney disease” may refer to a group that does not yet have kidney disease but has risk factors for kidney disease.

In the present specification, “stage 1 kidney disease” may refer to a stage where CKD is diagnosed through medical history of a normal or near-normal glomerular filtration rate (GFR), a persistent decline in GFR, imaging tests, or the like.

Another aspect provides a kit for diagnosing kidney disease, the kit including the composition.

The composition is the same as described herein.

The kit may provide, based on the IRIS guidelines for staging CKD, information on the onset of a chronic kidney risk group (a stage with risk factors), Stage 1 CKD (IRIS stage 1), or Stages 2 to 4 CKD (IRIS stages 2 to 4). In addition, the kit may be capable of distinguishing the stages based on the IRIS guidelines for staging CKD.

The kit may be applicable to various types of diagnostic kits utilizing antigen-antibody reactions, such as lateral flow assay or indirect immunofluorescence assay, but is not limited thereto.

The kit may include not only preparations for measuring the expression level of the protein or the gene, but also tools, reagents, etc. commonly used in immunological assays.

Examples of the tools or reagents may include a suitable carrier, a labeling substance capable of generating a detectable signal, a chromophore, a solubilizer, a detergent, a buffer, a stabilizer, etc., but are not limited thereto. When the labeling substance is an enzyme, a substrate and a reaction stopper that can measure the enzyme activity may be also included. The carrier may be a water-soluble carrier or a water-insoluble carrier. An example of the water-soluble carrier may include a physiologically acceptable buffer known in the art, such as PBS, and examples of the water-insoluble carrier may include polystyrene, polyethylene, polypropylene, polyester, polyacrylonitrile, a fluorocarbon resin, cross-linked dextran, polysaccharide, a polymer such as magnetic fine particle plated with metal on latex, other paper, glass, metal, agarose, and a combination thereof.

The kit may include a sample pad, a conjugate pad, a stacking pad, a membrane, an absorbent pad, and a solid support (e.g., a backing card), but is not limited thereto.

The “sample pad” may refer to a pad for receiving a sample to be analyzed and enabling diffusive flow, and it consists of a material having sufficient porosity to receive and contain a sample to be analyzed. Such a porous material may include fibrous paper, a microporous membrane made of cellulose materials, cellulose, a cellulose derivative such as cellulose acetate, nitrocellulose, glass fiber, a fabric such as naturally occurring cotton and nylon, or a porous gel, but is not limited thereto.

The “conjugate pad” may refer to a pad receiving a sample that is diffusively transferred from the sample pad. The conjugate pad may be composed of a material capable of diffusive flow, similar to the sample pad, but is not limited thereto.

The “membrane” may be a test line formed to trap analytes within a sample, and may be made of any material that the sample material can pass through. For example, the membrane may be formed from: naturally occurring materials, synthetic materials, or naturally occurring materials that have been modified by synthesis, such as polysaccharides (e.g., cellulose derivatives such as cellulose materials, paper, cellulose acetate, and nitrocellulose); polyether sulfones; polyethylene; nylon; polyvinylidene fluoride (PVDF); polyester;

polypropylene; silica; inorganic materials uniformly dispersed in a porous polymer matrix with polymers such as vinyl chloride, a vinyl chloride-propylene copolymer, and a vinyl chloride-vinyl acetate copolymer, such as inert alumina, diatomite, MgSO₄, or other inorganic fine substances; naturally occurring fabric (e.g., cotton) and synthetic fabric (e.g., nylon or rayon); porous gels such as silica gel, agarose, dextran, and gelatin; polymer films such as polyacrylamide; and the like. In an embodiment, the membrane may be a nitrocellulose (NC) membrane, a glass fiber membrane, a polyethersulfone (PES) membrane, a cellulose membrane, a nylon membrane, or a combination thereof, but is not limited thereto.

The membrane may have a test region and a control region that are formed sequentially from the conjugated pad in the direction of the absorbent pad, but is not limited thereto.

The “absorbent pad” may be positioned adjacent to or near the end of the membrane. The absorbent pad may typically receive a fluid sample that moves across the entire membrane. The absorbent pad may help promote capillary action and diffusive flow of fluid through the membrane.

The solid support may be formed of any material as long as it can support and transport the aforementioned sample pad, conjugate pad, membrane, stacking pad, and absorption pad. In an embodiment, the support may be liquid impermeable such that fluid of a sample diffusing through the membrane does not leak through the support. Examples of the support may include: glass; polymeric materials such as polystyrene, polypropylene, polyester, polybutadiene, polyvinyl chloride, polyamide, polycarbonate, epoxide, methacrylate, polymelamine, etc.; and the like, but are not limited thereto.

The kit may include the aforementioned sample pad, conjugated pad, stacking pad, membrane, and absorbent pad that are disposed sequentially on the same solid support.

Another aspect provides a method of providing information for diagnosis of kidney disease, the method including: measuring an expression level of a neutrophil gelatinase-associated lipocalin (NGAL) protein, a kidney injury molecule-1 (KIM-1) protein, or a combination thereof, or a gene encoding the same, in a biological sample obtained from a subject; and comparing the measured expression level with expression levels of proteins or a combination thereof, or a gene encoding the same, in a normal group.

In an embodiment, the method may further include measuring and setting, as an independent variable, an expression level of one or more proteins selected from the group consisting of SDMA, serum creatinine (sCr), inorganic phosphorus, amylase, and BUN, or a gene encoding the same.

The method may further include: setting, as an independent variable, the measured expression level of the one or more proteins or the gene, and setting, as a dependent variable, the onset of a CKD risk group (i.e., a stage with risk factors), Stage 1 CKD (IRIS stage 1), or Stages 2 to 4 CKD (IRIS stages 2 to 4) based on IRIS guidelines for staging CKD; modeling the relation between the independent variable and the dependent variable by logistic regression analysis to deduce a model equation; and determining the subject to belong to the risk group of kidney disease or to be at Stage 1 CKD (IRIS stage 1) or Stages 2 to 4 CKD (IRIS stages 2 to 4), when a value deduced by the model equation is greater than or equal to a predetermined cutoff value.

In an embodiment, the logistic regression analysis is a binary algorithm for modeling the relation between data on markers associated with kidney disease and the risk group of kidney disease (stage with risk factors), Stage 1 CKD (IRIS stage 1), or Stages 2 to 4 CKD (IRIS stages 2 to 4), wherein the basic formula is as follows:

$$log ( \frac{p}{1 - p} ) = {β}_{0} + {β}_{1} {X}_{1} + {β}_{2} {X}_{2} + … + {β}_{η} {X}_{\text{?}}$$

$$\text{?} \text{indicates text missing or illegible when filed}$$

Here, in the basic formula, the dependent variable P is the probability value of belonging to the CKD risk group (stage with risk factors) or being at Stage 1 CKD (IRIS stage 1) or Stages 2 to 4 CKD (IRIS stages 2 to 4), the dependent variable 1-P is the probability value of being normal, and corresponding to normal, and the independent variables X1 to Xn are the variables for markers associated with kidney disease in the subject.

The markers associated with kidney disease in the subject may be the concentration of NGAL, KIM-1, SDMA, creatinine, inorganic phosphorus, amylase, and BUN, substituted for the corresponding units.

That is, using the markers associated with kidney disease in the subject, the relation of the probability values for belonging to the risk group of kidney disease (stage with risk factors), the Stage 1 CKD (IRIS stage 1) or being at Stages 2 to 4 CKD (IRIS stages 2 to 4) may be modelled by logistic regression analysis to deduce estimated coefficients of the basic equation to establish a model equation.

In an embodiment, the model equation may be the formula with the highest explanatory power (pseudo R-squared; R2) in logistic regression analysis.

In an embodiment, the cutoff value may be determined by converting the point on the receiver operating characteristic (ROC) curve, where “sensitivity×specificity” shows a maximum value according to a concordance probability method.

In an embodiment, the model equation may be any one selected from Equations 1 to 6 below:

$$RNK ( y ) = 1.648 \times pNGAL ( ng / mL ) + 3.287 \times pKIM - 1 ( ng / ml ) - 12.2 \qquad [ Equation 1 ]$$

$$RNKC ( y ) = 1.71 \times pNGAL ( ng / ml ) + 3.306 \times pKIM - 1 ( ng / mL ) + 0.9716 \times sCr ( mg / dl ) - 13.22 \qquad [ Equation 2 ]$$

$$RNKS ( y ) = 1.928 \times pNGAL ( ng / ml ) + 3.948 \times pKIM - 1 ( ng / ml ) + 0.4207 \times SDMA ( μg / dl ) - 19.09 \qquad [ Equation 3 ]$$

$$RNKA = 1.398 \times pNGAL ( ng / ml ) + 3.989 \times pKIM - 1 ( ng / ml ) + 0.5979 \times Age ( year ) - 17.02 \qquad [ Equation 4 ]$$

$$RNKR = 2.832 \times pNGAL ( ng / ml ) + 4.726 \times pKIM - 1 ( ng / ml ) + 8.756 \times CRP ( mg / dl ) - 21.36 \qquad [ Equation 5 ]$$

$$RNKCS = 2.15 \times pNGAL ( ng / ml ) + 4.178 \times pKIM - 1 ( ng / ml ) + 1.798 \times sCr ( mg / dl ) + 0.4377 \times SDMA ( μg / dl ) - 21.97 \qquad [ Equation 6 ]$$

In an embodiment, the cutoff value is determined such that a value derived from any one selected from Equations 1 to 6 exceeds a level found in a normal subject not having kidney disease, and if the derived value is greater than or equal to the cutoff value, it means that the kidneys are damaged and the subject has kidney disease or is at risk of kidney disease. Specifically, it means that the subject has risk factors for CKD or is at Stages 1 to 4 CKD (IRIS stages 1 to 4) based on the IRIS guidelines for staging CKD.

In a preferred embodiment, the cutoff value (RNK) of Equation 1 may range from any number selected from −1.94 to 1.58, preferably any number selected from −1.50 to 1.58, more preferably any number selected from 0 to 1.58, and more preferably any number selected from 1.40 to 1.58, and may be more preferably 1.58.

In a preferred embodiment, the cutoff value (RNKC) of Equation 2 may range from any number selected from −2.02 to 1.49, preferably any number selected from −1.00 to 1.49, more preferably any number selected from 0.00 to 1.49, and more preferably any number selected from 1.26 to 1.49, and may be more preferably 1.49.

In a preferred embodiment, the cutoff value (RNKS) of Equation 3 may range from any number selected from −3.57 to 2.09, preferably any number selected from −0.00 to 2.09, and more preferably any number selected from 1.93 to 2.09, and may be more preferably 2.085.

In a preferred embodiment, the cutoff value (RNKA) of Equation 4 may range from any number selected from −3.33 to 0.54, preferably any number selected from −2.77 to 0.53, more preferably any number selected from −0.36 to 0.52, and more preferably any number selected from 0.00 to 0.51, and may be more preferably 0.51.

In a preferred embodiment, the cutoff value (RNKR) of Equation 5 may range from any number selected from −3.49 to −0.04 and preferably any number selected from −0.30 to −0.05, and may be more preferably −0.077.

In a preferred embodiment, the cutoff value (RNKCS) of Equation 6 may range from any number selected from −4.27 to 2.50, preferably any number selected from −2.35 to 2.40, more preferably any number selected from 2.03 to 2.30, and more preferably any number selected from 2.10 to 2.20, and may be more preferably 2.11.

In an embodiment, one specific example, any one of the values derived from Equations 1 to 6 may be transformed using an exponential function to converge a value of 1 or 0. Here, the prediction equation using the exponential function to classify the risk group of kidney disease and IRIS stages 1 to 4 (indicating a value of 1) and normal people (indicating a value of 0) is as shown in Equation 7. In Equation 7, the p value is a percentage with a distribution ranging from 0 to 1, wherein a value of less than 0.5 is determined as having no risk of kidney disease, and a value of 0.5 or more is determined as belonging to the risk group of kidney disease or a group at higher stages.

$$p = exp ( y ) / [ exp ( y ) + 1 ] \qquad [ Equation 7 ]$$

In the equation above, y is a value derived from Equations 1 to 6.

In an embodiment, the model equation may be any one selected from Equations 8 to 13 below:

$$SNK ( y ) = 0.5541 \times pNGAL ( ng / ml ) + 0.3766 \times pKIM - 1 ( ng / ml ) - 2.614 \qquad [ Equation 8 ]$$

$$SNKC ( y ) = 0.5522 \times pNGAL ( ng / ml ) + 0.3146 \times pKIM - 1 ( ng / ml ) + 0.4417 \times sCr ( mg / dl ) - 2.792 \qquad [ Equation 9 ]$$

$$SNKS ( y ) = 0.442 \times pNGAL ( ng / mL ) + 0.001992 \times pKIM - 1 ( ng / ml ) + 0.2562 \times SDMA ( μg / dl ) - 4.079 \qquad [ Equation 10 ]$$

$$SNKA ( y ) = 0.447 \times pNGAL ( ng / ml ) + 0.2079 \times pKIM - 1 ( ng / mL ) + 0.2108 \times Age ( year ) - 3.55 \qquad [ Equation 11 ]$$

$$SNKP ( y ) = 0.4599 \times pNGAL ( ng / ml ) + 0.3363 \times pKIM - 1 ( ng / ml ) + 1.004 \times Inorganic phosphorus ( mg / dl ) - 5.678 \qquad [ Equation 12 ]$$

$$SNKCS ( y ) = 0.4406 \times pNGAL ( ng / ml ) + 0.007931 \times pKIM - 1 ( ng / ml ) - 0.07766 \times sCr ( mg / dl ) + 0.258 \times SDMA ( μg / dl ) - 4.048 \qquad [ Equation 13 ]$$

In an embodiment, the cutoff value is determined such that a value derived from any one selected from Equations 8 to 13 exceeds a level found in a normal subject not having kidney disease or a subject corresponding to the risk group for kidney disease, and if the derived value is greater than or equal to the cutoff value, it means that the GFR is normal or near-normal but the subject has kidney damage and therefore has kidney diseases. Specifically, it means that the subject is at Stages 1 to 4 CKD (IRIS stages 1 to 4) based on the IRIS guidelines for staging CKD. The cutoff value for a value derived from any one selected from Equations 8 to 13 for determining IRIS stages 1 to 4 of the present invention may be any number selected from −1.67 to 4.26.

In a preferred embodiment, the cutoff value (SNK) of Equation 8 may range from any number selected from −0.74 to 4.40, preferably any number selected from 0.00 to 4.00, and more preferably any number selected from 0.30 to 1.00, and may be more preferably 0.89.

In a preferred embodiment, the cutoff value (SNKC) of Equation 9 may range from any number selected from −0.70 to 4.26, and may be more preferably 0.83.

In a preferred embodiment, the cutoff value (SNKS) of Equation 10 may range from any number selected from −1.25 to 3.30, and may be more preferably 0.49.

In a preferred embodiment, the cutoff value (SNKA) of Equation 11 may range from any number selected from −1.67 to 4.10, and may be more preferably 0.96.

In a preferred embodiment, the cutoff value (SNKP) of Equation 12 may range from any number selected from −0.76 to 3.65, and may be more preferably 0.76.

In a preferred embodiment, the cutoff value (SNKCS) of Equation 13 may range from any number selected from −1.26 to 3.30, and may be more preferably 0.48.

In an embodiment, one specific example, any one of the values derived from Equations 8 to 13 may be transformed using an exponential function to converge a value of 1 or 0. Here, the prediction equation using the exponential function to classify the Stages 1 to 4 CKD (IRIS stages 1 to 4, indicating a value of 1) and normal people and the risk group of kidney disease (indicating a value of 0) is as shown in Equation 14. In Equation 14, the p value is a percentage with a distribution ranging from 0 to 1, wherein a value of less than 0.5 is determined as having no risk of kidney disease or being at the risk group (a stage with risk factors), and a value of 0.5 or more is determined as being at Stages 1 to 4 CKD.

$$p = exp ( y ) / [ exp ( y ) + 1 ] \qquad [ Equation 14 ]$$

In the equation above, y is a value derived from Equations 8 to 13.

In an embodiment, the model equation may be any one selected from Equations 15 to 23 below:

$$TNK ( y ) = 0.03031 \times pNGAL ( ng / ml ) + 1.187 \times pKIM - 1 ( ng / ml ) - 5.538 \qquad [ Equation 15 ]$$

$$TNKC ( y ) = - 0.01621 \times pNGAL ( ng / ml ) + 0.9737 \times pKIM - 1 ( ng / ml ) + 3.773 \times sCr ( mg / dl ) - 8.309 \qquad [ Equation 16 ]$$

$$TNKS ( y ) = - 0.01627 \times pNGAL ( ng / ml ) + 0.631 \times pKIM - 1 ( ng / ml ) + 0.4914 \times sCr ( mg / dl ) - 10.55 \qquad [ Equation 17 ]$$

$$TNKA ( y ) = 0.02812 \times pNGAL ( ng / ml ) + 1.078 \times pKIM - 1 ( ng / ml ) + 0.2033 \times Age ( year ) - 7.344 \qquad [ Equation 18 ]$$

$$TNKP ( y ) = - 0.1125 \times pNGAL ( ng / ml ) + 1.42 \times pKIM - 1 ( ng / ml ) + 0.7162 \times Inorganic phosphorus ( mg / dl ) - 8.453 \qquad [ Equation 19 ]$$

$$TNKAm ( y ) = - 0.05275 \times pNGAL ( ng / ml ) + 1.001 \times pKIM - 1 ( ng / ml ) + 0.001492 \times Amylase ( U / L ) - 5.468 \qquad [ Equation 20 ]$$

$$TNKCS ( y ) = - 0.05199 \times pNGAL ( ng / ml ) + 0.2173 \times pKIM - 1 ( ng / mL ) + 9.823 \times sCr ( mg / dl ) + 0.9584 \times SDMA ( μg / dl ) - 26.57 \qquad [ Equation 21 ]$$

$$TNKB ( y ) = - 0.0266 \times pNGAL ( ng / ml ) + 1 \times pKIM - 1 ( ng / ml ) + 0.11 \times BUN ( mg / dl ) - 7.155 \qquad [ Equation 22 ]$$

$$TNKCSB ( y ) = - 0.08838 \times pNGAL ( ng / ml ) + 0.2262 \times pKIM - 1 ( ng / ml ) + 8.739 \times sCr ( mg / dl ) + 0.961 \times SDMA ( μg / dl ) + 0.04618 \times BUN ( mg / dL ) - 26.28 \qquad [ Equation 23 ]$$

In an embodiment, the cutoff value is determined such that a value derived from any one selected from Equations 15 to 23 exceeds a level found in a normal subject not having kidney disease or a subject corresponding to the risk group or IRIS stage 1, and if the derived value is greater than or equal to the cutoff value, it means that the subject has kidney damage and therefore has kidney diseases. Specifically, it means that the subject is at Stages 2 to 4 CKD (IRIS stages 2 to 4) based on the IRIS guidelines for staging CKD. The cutoff value for a value derived from any one selected from Equations 15 to 23 for determining IRIS stages 2 to 4 of the present invention may be any number selected from −5.17 to 2.23.

In a preferred embodiment, the cutoff value (TNK) of Equation 15 may range from any number selected from −5.17 to −4.08, and may be more preferably −4.83.

In a preferred embodiment, the cutoff value (TNKC) of Equation 16 may range from any number selected from −3.72 to 1.31, and may be more preferably −0.14.

In a preferred embodiment, the cutoff value (TNKS) of Equation 17 may range from any number selected from −3.57 to −0.05, and may be more preferably −0.08.

In a preferred embodiment, the cutoff value (TNKA) of Equation 18 may range from any number selected from −4.28 to 1.65, and may be more preferably −0.30.

In a preferred embodiment, the cutoff value (TNKP) of Equation 19 may range from any number selected from −1.78 to 1.31, and may be more preferably −0.52.

In a preferred embodiment, the cutoff value (TNKAm) of Equation 20 may range from any number selected from −2.12 to 1.37, and may be more preferably −0.51.

In a preferred embodiment, the cutoff value (TNKCS) of Equation 21 may range from any number selected from −0.76 to 2.23, and may be more preferably −0.76.

In a preferred embodiment, the cutoff value (TNKB) of Equation 22 may range from any number selected from −4.18 to 1.73, and may be more preferably 0.13.

In a preferred embodiment, the cutoff value (TNKCSB) of Equation 23 may range from any number selected from −1.66 to 1.51, and may be more preferably −0.52.

In an embodiment, one specific example, any one of the values derived from Equations 15 to 23 may be transformed using an exponential function to converge a value of 1 or 0. Here, the prediction equation using the exponential function to classify Stages 2 to 4 CKD (IRIS stages 2 to 4, indicating a value of 1) and normal people and the risk group of kidney disease (a stage with risk factors) and Stage 1 CKD (IRIS stage 1) is as shown in Equation 24. In Equation 24, the p value is a percentage with a distribution ranging from 0 to 1, wherein a value of less than 0.5 is determined as being at Stage 1 or less of kidney disease, and a value of 0.5 or more is determined as being at Stages 2 or higher of kidney disease.

$$p = exp ( y ) / [ exp ( y ) + 1 ] \qquad [ Equation 24 ]$$

In the equation above, y is a value derived from Equations 15 to 23.

The method of providing information according to the present invention can distinguish between a normal group and a CKD risk group with a sensitivity of 90% or higher and a specificity of 95% or higher, when the dependent variable is the CKD risk group (a stage with risk factors). Therefore, the present invention may enable early detection of kidney dysfunction in a subject, particularly the CKD risk group (a stage with risk factors), and prompt treatment.

The method of providing information according to the present invention can distinguish between a normal group, the CKD risk group, and IRIS stage 1, with a sensitivity of 85% or higher and a specificity of 90% or higher, when the dependent variable indicates the onset of IRIS stage 1. Therefore, the present invention may enable early detection of kidney dysfunction in a subject, particularly Stage 1 CKD (IRIS stage 1), and prompt treatment.

In addition, the method of providing information according to the present invention can distinguish between a normal group, the CKD risk group (a stage with risk factors), Stage 1 CKD (IRIS stage 1), and IRIS stages 2 to 4, with a sensitivity of 90% or higher and a specificity of 90% or higher, when the dependent variable indicates the onset of IRIS stages 2 to 4.

Another aspect provides a method of diagnosing kidney disease, the method including: measuring an expression level of a neutrophil gelatinase-associated lipocalin (NGAL) protein, a kidney injury molecule-1 (KIM-1) protein, or a combination thereof, or a gene encoding the same, in a biological sample obtained from a subject; and comparing the measured expression level with expression levels of proteins or a combination thereof, or a gene encoding the same, in a normal group.

Another aspect provides a method of treating kidney disease, the method including: measuring an expression level of a neutrophil gelatinase-associated lipocalin (NGAL) protein, a kidney injury molecule-1 (KIM-1) protein, or a combination thereof, or a gene encoding the same, in a biological sample obtained from a subject.

Another aspect provides a system for diagnosing kidney disease, including: an input unit configured to input a concentration of at least one marker selected from NGAL KIM-1 as measured from a body fluid sample of a subject, or to input, together with the concentration of the at least one marker, a concentration of at least one marker selected from symmetric dimethylarginine (SDMA), creatinine, inorganic phosphorus, amylase, and BUN; a variable setting unit configured to set, as a single or multiple independent variable, the input concentration of the at least one marker, and to set, as a dependent variable, the onset of a chronic kidney risk group (a stage with risk factors), Stage 1 CKD (IRIS stage 1), or Stages 2 to 4 CKD (IRIS stages 2 to 4) based on the IRIS guidelines for staging CKD; an inference engine unit configured to model the relation between the multiple independent variable and the dependent variable by logistic regression analysis to deduce a model equation; and a diagnosis unit configured to determine a subject to belong to a risk group of kidney disease or to be at Stage 1 CKD (IRIS stage 1) or Stages 2 to 4 CKD (IRIS stages 2 to 4), when a value deduced by the model equation is greater than or equal to a predetermined cutoff value, wherein the value is deduced by substituting data for the at least one marker input by the input unit into the independent variable of the inferred model equation.

Another aspect provides a computer-readable recording medium having recorded thereon a computer program for executing the method on a computer.

The recording medium may be implemented as an application (or a program) and readable by a terminal device (or a computer). Specifically, the recording medium may include any type of recording device or medium on which data that can be read by a computing system is stored.

### Advantageous Effects of Invention

According to the present invention, kidney disease or risk of kidney disease can be diagnosed early with greater accuracy, sensitivity, and specificity. In particular, according to the present invention, based on the International Renal Interest Society (IRIS) guidelines for staging chronic kidney disease (CKD), it is possible to distinguish between a normal group and a CKD risk group (a stage with risk factors), Stage 1 CKD (IRIS stage 1), or Stages 2 to 4 CKD (IRIS stages 2 to 4), with a sensitivity of 85% or higher and a specificity of 90% or higher.

### BRIEF DESCRIPTION OF DRAWINGS

FIG. 1 shows analysis results of a scatter plot with log scale for the correlation between plasma concentrations of NGAL (pNGAL) and KIM-1 (pKIM-1) in companion dogs and concentrations of conventional biomarkers, creatinine (Cr) and SDMA.

FIG. 2 shows results of statistical analysis of the scatter plot of FIG. 1 that has been converted to logs.

FIG. 3 shows results of receiver operating characteristic (ROC) curve analysis in a risk group of kidney disease or IRIS stages 1 to 4 for evaluating diagnostic accuracy.

FIG. 4 shows results of ROC curve analysis in IRIS stages 1 to 4 for evaluating diagnostic accuracy by baseline stage of disease state.

FIG. 5 shows results of ROC curve analysis in IRIS stages 2 to 4 for evaluating diagnostic accuracy by baseline stage of disease state.

FIG. 6A is a schematic diagram simply illustrating a structure of a kit for diagnosing kidney disease according to an embodiment of the present invention, and FIG. 6B is a simplified scheme of the operating principle of the kit for diagnosing kidney disease according to an embodiment of the present invention.

### DETAILED DESCRIPTION

Hereinafter, preferable examples are presented to help understanding of the present invention. However, examples below are only presented only for easier understanding of the present invention, and the contents of the present invention are not limited by these examples. Since examples may apply various modifications, examples are not limited to those described herein and may be implemented in various forms.

### Example 1. Diagnosis of Kidney Disease

### 1.1. Selection of Experimental Subjects

Plasma samples and medical records of dogs brought into the Animal Hospital affiliated to Konkuk University from Jul. 1, 2018 to Aug. 31, 2022 were used. To minimize the influence of dog weight and breed during the study and to reflect the characteristics of mainly raising small dogs in Korea, only small dogs weighing less than 10 kg were included in the study. Additionally, plasma samples with insufficient volume for analysis or those with hemolysis were excluded from the experimental subjects, in consideration of affecting the results of enzyme-linked immunosorbent assay. Next, among the small dog population, dogs that had been tested for plasma SDMA, BUN, and plasma creatinine (Cr) levels were included. Finally, dogs that did not undergo SDMA testing but visited for a general checkup or mild lameness without any major abnormalities in clinical symptoms or clinicopathological test results and without any long-term medication or underlying disease were regarded as a normal control group.

### 1.2. Sample Treatment and Biomarker Analysis

Plasma samples remaining after clinical examination of dogs that satisfied the conditions for selecting experimental subjects in Example 1.1 were analyzed for concentrations of pNGAL and pKIM-1.

Specifically, venous blood collected in a lithium-heparin tube was centrifuged at 3,000 rpm for 6 minutes at room temperature to separate the plasma. The levels of creatinine, BUN, SDMA, inorganic phosphorous, and amylase levels in the plasma were measured by using a Catalyst One chemistry analyzer (IDEXX Laboratories), immediately after sample collection. Afterwards, the remaining plasma samples were frozen within 6 hours and stored at −78° C., the concentration of SDMA in the control group was measured with a Catalyst One analyzer using the frozen plasma samples.

The concentrations of pNGAL and pKIM-1 were measured by using a dog-specific sandwich enzyme-linked immunosorbent assay (ELISA) kits (ab205084 and ab205085, respectively; Abcam, Cambridge, UK) according to the manufacturer's instructions. For this purpose, the freshly frozen plasma samples were quickly thawed in a water bath at 37° C. Thereafter, the plasma samples were diluted with a diluent at a ratio of 1:10 to measure the concentration of pKIM-1, and the plasma samples were eluted with a diluent at a ratio of 1:50 to measure the concentration of pNGAL. Next, the absorbance of the diluted samples was measured at 450 nm by using a microplate reader (Tecan, Zurich, Switzerland) according to the manufacturer's instructions. Next, the concentrations of pNGAL and pKIM-1 were calculated from a standard curve (4-parameter logistic curve) calculated from the absorbance measurements of standard substances.

### 1.3. Staging and Grouping of Kidney Disease

The study population selected under the conditions of Example 1.1 was classified into four stages based on the IRIS guidelines for staging CKD published in 2019. For reference, the risk group consisted of subjects who had risk factors for kidney disease and thus did not fulfil the conditions of the control group, but had the plasma creatinine and SDMA concentrations in a normal range. The basis for determining the risk group include myxomatous mitral valve disease (MMVD), portosystemic shunt, chronic heart failure, hyperadrenocorticism (HAC), diabetes mellitus, babesiosis, and the like. Underlying diseases including MMVD and HAC were identified using medical records such as physical examination, clinicopathological analysis, and/or radiological examination. In addition, since the number of subjects in Stage 4 was significantly small, these subjects were combined into groups at Stages 3 and 4. Accordingly, 15 dogs were classified into the risk group, 43 dogs into IRIS stage 1, 33 dogs into stage 2, and 16 dogs into stages 3 and 4.

### 1.4. Statistical Analysis and Derivation of Optimal Model

Statistical analysis and deviation of optimal diagnostic model were performed by using GraphPad Prism (version 9.3.1; GraphPad Software, San Diego, CA, USA).

Categorical variables, such as gender and physical status scores, were analyzed by using a chi-square test. Here, the age of a patient was converted to a decimal point based on the date of sample collection and considered as a continuous variable. Differences between the four groups in variables that followed a normal distribution, including log-transformed data, were analyzed by using one-way ANOVA followed by post-hoc analysis such as Bonferroni.

To derive an optimal diagnostic model, the extent of influence of multiple independent variables on the dependent variable was confirmed by simple logistic regression analysis (or crude logistic regression analysis), and only the variables that were significant at a significance level of 0.05 were combined, and an equation was derived by modeling through multiple logistic regression analysis.

The diagnostic accuracy of the four biomarkers of kidney disease and the derived model was analyzed by using receiver operating characteristics (ROC) curves, and the optimal value from the concordance probability method (“sensitivity x specificity”) was used to select the optimal cutoff value.

### Experimental Example 1. Comparison of Existing Biomarker with NGAL and KIM-1

The plasma concentrations of SDMA and creatinine in companion dogs were analyzed by comparison with the concentrations of NGAL and KIM-1, and the correlation with the concentrations measured in normal companion dogs without kidney disease (a control group) was also analyzed.

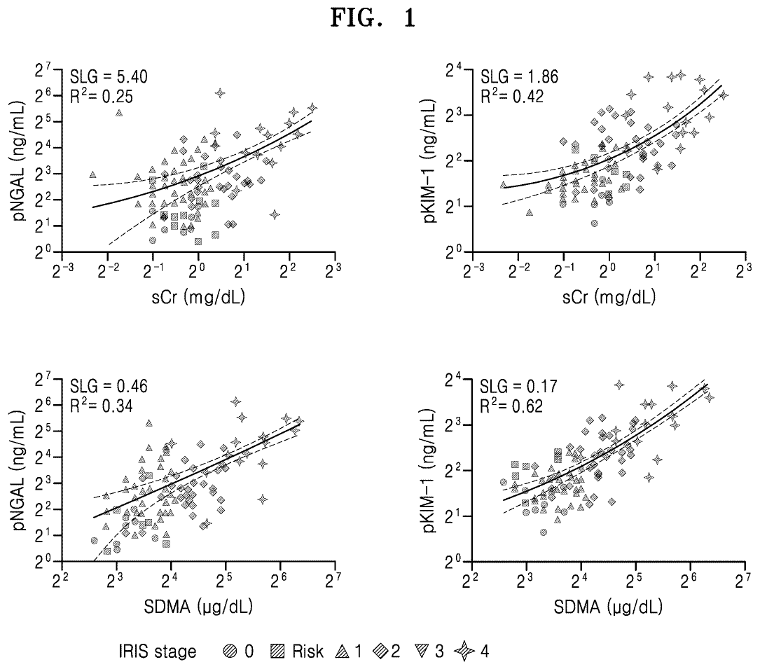

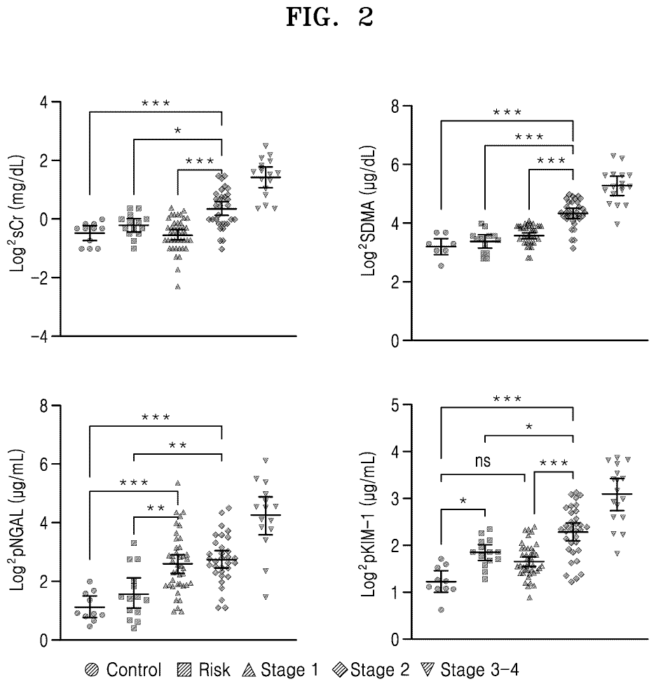

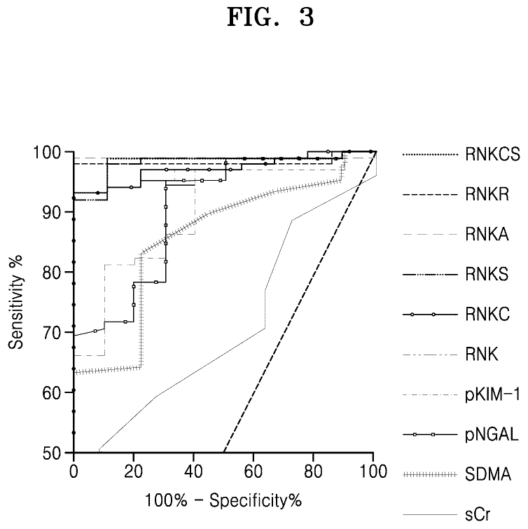

Specifically, the equivalence and difference between the plasma concentrations of NGAL (pNGAL) and KIM-1 (pKIM-1) and the concentrations of conventional biomarkers, creatinine (Cr) and SDMA, were analyzed on a log scale via a scatter plot (FIGS. 1 and 2).

FIG. 1 shows a scatter plot of the correlation between the plasma concentrations of NGAL (pNGAL) and KIM-1 (pKIM-1) in companion dogs and the concentrations of conventional biomarkers, Cr and SDMA, and FIG. 2 shows the scatter plot of FIG. 1 converted to a log scale to show the difference in the concentration of biomarkers by kidney disease.

As shown in FIGS. 1 and 2, it was confirmed that pNGAL and pKIM-1 of the present invention did not show significant differences from the conventional biomarkers, sCr and SDMA, at IRIS stages 3 and 4, but did show significant differences in the risk group and at IRIS stage 1. In particular, the conventional biomarkers did not show a significant difference between the normal control group and the risk group, whereas pNGAL and pKIM-1 of the present invention showed a significant difference.

These results imply that pNGAL and pKIM-1 of the present invention can be used as biomarkers for the early diagnosis of kidney disease that can distinguish between the normal control group and the risk group or IRIS stage 1, which could not be distinguished using the conventional biomarkers.

### Experimental Example 2. Optimal Model for Diagnosing Risk Group Of Kidney Disease

The biomarkers that showed statistical significance in both the normal group and the risk group of kidney disease were combined to provide meaningful information, and an algorithm that can distinguish the differences between the two groups in an equation was invented. The mathematical model used in the present invention is based on binary logistic regression analysis. The inventors of the present invention were able to obtain various regression analysis models by using different combinations of biomarkers, and confirmed the feasibility of technology for early diagnosis of kidney damage or kidney disease by showing high sensitivity and specificity in distinguishing between the normal group and the risk group of kidney disease.

### Experimental Example 2.1. Derivation of Diagnostic Model for Risk Group of Kidney Disease-Distinguishing of Normal Group Vs. Risk Group and Iris Stages 1 to 4

Optimal indexes for distinguishing of the risk group of kidney disease (normal group vs. risk group and IRIS stages 1 to 4) were to be derived. Specifically, markers associated with kidney disease (including chemical test results or clinical index) were set as multiple independent variables, and the risk group or a group at higher stages was set as a dependent variable. Then, by crude logistic regression analysis of the Graphpad Prism program, the extent of influence of the multiple independent variables on the dependent variable was confirmed and only the variables that were significant at a significance level of 0.05 were combined (Table 1). As a result of the crude analysis, the dependent variables, except for sCR and BUN, were correlated with the independent variables including NGAL, KIM-1, SDMA, and CRP, and thus the optimal combination was set using these six variables.

TABLE 1

| | Crude Logistic regression analysis | | | | | | |
|---|---|---|---|---|---|---|---|
| | | | | | | | Pseudo |
| Division | n | OR | SE | z | p | (95% CI) | R2 |
| NGAL | 112 | 2.983 | 1.048 | 2.766 | 0.0057 | 1.598~7.711 | 0.2372 |
| KIM-1 | 116 | 13.65 | 2.22 | 3.042 | 0.0024 | 3.337~101.9 | 0.2766 |
| SDMA | 115 | 1.475 | 1.506 | 2.649 | 0.0081 | 1.157~2.063 | 0.143 |
| sCr | 117 | 7.241 | 0.8588 | 1.881 | 0.06 | 1.423~81.73 | 0.04226 |
| Age | 117 | 1.51 | 0.7133 | 3.958 | <0.0001 | 1.257~1.905 | 0.2661 |
| BW | 116 | 1.147 | 0.9324 | 0.6453 | 0.5187 | 0.7729~1.797 | 0.002732 |
| BP | 105 | 1.042 | 3.378 | 1.545 | 0.1223 | 0.9908~1.101 | 0.02565 |
| BUN | 117 | 1.071 | 0.6869 | 1.866 | 0.062 | 1.011~1.167 | 0.04213 |
| BCR | 117 | 1.015 | 0.6628 | 0.5672 | 0.5706 | 0.9737~1.078 | 0.002341 |
| Globulin | 103 | 7.132 | 2.588 | 2.566 | 0.0103 | 1.836~39.46 | 0.07069 |
| Amylase | 45 | 1.005 | 1.889 | 1.488 | 0.1366 | 1~1.014 | 0.07687 |
| CRP | 57 | 78.94 | 0.7484 | 2.022 | 0.0432 | 2.768~11689 | 0.1738 |
| Inorganic | 68 | 3.219 | 2.117 | 1.783 | 0.0746 | 1.129~15.85 | 0.0841 |
| Phosphorus | | | | | | | |

Crude: Univariate logistic, Simple logistic
*p < .05
**p < .01
***p < .001

In the top entry of the table, n represents the number of observations, S.E. represents a standard error, 95% CI (confidence interval) represents the lower and upper limits of the 95% confidence interval of the estimated coefficient, z refers to a t-distribution statistic obtained by dividing the estimated coefficient by the S.E., p refers to a test statistic for the extent to which the null hypothesis can be rejected, and Pseudo R2 refers to the extent to which the dependent variable is explained by the independent variables, i.e., the explanatory power (predictive power).

The functional relation between the combined independent and dependent variables was modeled through multiple logistic regression analysis of the Graphpad Prism program to deduce an equation. Here, the logistic regression analysis is a binary algorithm for modeling the relation between data on risk factors and the risk of kidney disease.

By such modeling, RNK, RNKC, RNKS, RNKA, RNKR and RNKCS as the optimal models (functions) with the highest pseudo R2 (referring to explanatory power, the intensity of the relation between the dependent and independent variables) were derived (Tables 2 to 7). For reference, RNK is a function using pNGAL and pKIM-1, RNKC is a function using pNGAL, pKIM-1, and sCr, RNKS is a function using pNGAL, pKIM-1, and SDMA, RNKA is a function using pNGAL, pKIM-1, and age, RNKR is a function using pNGAL, pKIM-1, and CRP, and RNKCS is a function using pNGAL, pKIM-1, sCr, and SDMA.

TABLE 2
Model 1: RNK

| | Crude | | | | | |
|---|---|---|---|---|---|---|
| | | | | | | Pseudo |
| Division | Coef. | SE | z | p | (95% CI) | R2 |
| NGAL | 1.648 | 0.7615 | 2.163 | 0.0305 | 0.5359 | 0.5937 |
| | | | | | to 3.672 | |
| KIM-1 | 3.287 | 1.291 | 2.545 | 0.0109 | 1.300 | |
| | | | | | to 6.582 | |
| Constant | −12.2 | 4.796 | 2.543 | 0.011 | −24.96 | |
| | | | | | to −4.964 | |

n = 111,
LR chi2(2) = 37.09,
p < .0001

In the top entry of the table, Coef. represents a regression coefficient indicating the magnitude of the influence of the independent valuables on the dependent variable, S.E. represents a standard error, 95% CI (confidence interval) represents the lower and upper limits of the 95% confidence interval of the estimated coefficient, z refers to a t-distribution statistic obtained by dividing the estimated coefficient by the S.E., p refers to a test statistic for the extent to which the null hypothesis can be rejected, and Pseudo R2 refers to the extent to which the dependent variable is explained by the independent variables, i.e., the explanatory power (predictive power).

The estimated function equation from the logistic regression analysis of Model 1 (RNK) is as follows:

$$RNK ( y ) = 1.6 \; 48 \times pNGAL ( ng / mL ) + 3.287 \times pKIM - 1 ( ng / mL ) - 12. 2 \qquad [ Equation l ]$$

TABLE 3
Model 2: RNKC

| | Crude | | | | | |
|---|---|---|---|---|---|---|
| | | | | | | Pseudo |
| Division | Coef. | SE | z | p | (95% CI) | R2 |
| NGAL | 1.71 | 0.7944 | 2.153 | 0.0313 | 0.5571 | 0.5985 |
| | | | | | to 3.819 | |
| KIM-1 | 3.306 | 1.32 | 2.505 | 0.0122 | 1.277 | |
| | | | | | to 6.695 | |
| sCr | 0.9716 | 1.815 | 0.5353 | 0.5924 | −2.522 | |
| | | | | | to 5.061 | |
| Constant | −13.22 | 5.34 | 2.475 | 0.0133 | −27.22 | |
| | | | | | to −5.126 | |

n = 111,
LR chi2(3) = 37.39,
p < .0001

The estimated function equation from the logistic regression analysis of Model 2 (RNKC) is as follows:

$$RNKC ( y ) = 1.71 \times pNGAL ( ng / mL ) + 3.306 \times pKIM - 1 ( ng / mL ) + 0.9 \; 716 \times sCr ( mg / dl ) - 13.22 \qquad [ Equation 2 ]$$

TABLE 4
Model 3: RNKS

| | Crude | | | | | |
|---|---|---|---|---|---|---|
| | | | | | (95% | Pseudo |
| Division | Coef. | SE | z | p | CI) | R2 |
| NGAL | 1.928 | 0.9222 | 2.09 | 0.0366 | 0.5523 | 0.6716 |
| | | | | | to 4.312 | |
| KIM-1 | 3.948 | 1.529 | 2.582 | 0.0098 | 1.635 | |
| | | | | | to 7.900 | |
| SDMA | 0.4207 | 0.2544 | 1.653 | 0.0983 | 0.03526 | |
| | | | | | to 1.061 | |
| Constant | −19.09 | 7.196 | 2.653 | 0.008 | −37.42 | |
| | | | | | to −8.207 | |

n = 111,
LR chi2(3) = 41.95,
p < .0001

The estimated function equation from the logistic regression analysis of Model 3 (RNKS) is as follows:

$$RNKS ( y ) = 1.928 \times pNGAL ( ng / mL ) + 3.948 \times pKIM - 1 ( ng / mL ) + 0.4 \; 207 \times SDMA ( μg / dl ) - 19. 09 \qquad [ Equation 3 ]$$

TABLE 5
Model 4: RNKA

| | Crude | | | | | |
|---|---|---|---|---|---|---|
| | | | | | (95% | Pseudo |
| Division | Coef. | SE | z | p | CI) | R2 |
| NGAL | 1.398 | 0.7237 | 1.931 | 0.0534 | 0.2698 | 0.7906 |
| | | | | | to 3.557 | |
| KIM-1 | 3.989 | 1.791 | 2.227 | 0.0259 | 1.429 | |
| | | | | | to 8.754 | |
| Age | 0.5979 | 0.2533 | 2.361 | 0.0182 | 0.2158 | |
| | | | | | to 1.342 | |
| Constant | −17.02 | 6.612 | 2.574 | 0.01 | −34.94 | |
| | | | | | to −7.432 | |

n = 111,
LR chi2(3) = 49.39,
p < .0001

The estimated function equation from the logistic regression analysis of Model 4 (RNKA) is as follows:

$$RNKA = 1.398 \times pNGAL ( ng / mL ) + 3.9 \; 89 \times pKIM - 1 ( ng / mL ) + 0.5 \; 979 \times Age ( year ) - 17.02 \qquad [ Equation 4 ]$$

TABLE 6
Model 5: RNKR

| | Crude | | | | | |
|---|---|---|---|---|---|---|
| | | | | | (95% | Pseudo |
| Division | Coef. | SE | z | p | CI) | R2 |
| NGAL | 2.832 | 1.829 | 1.548 | 0.1216 | 0.4175 | 0.7447 |
| | | | | | to 7.871 | |
| KIM-1 | 4.726 | 2.613 | 1.809 | 0.0705 | 1.355 | |
| | | | | | to 12.84 | |
| CRP | 8.756 | 6.283 | 1.394 | 0.1634 | −0.4786 | |
| | | | | | to 25.23 | |
| Constant | −21.36 | 11.41 | 1.873 | 0.0611 | −55.48 | |
| | | | | | to −6.346 | |

n = 56,
LR chi2(3) = 31.42,
p < .0001

The estimated function equation from the logistic regression analysis of Model 5 (RNKR) is as follows:

$$RNKR = 2.832 \times pNGAL ( ng / mL ) + 4.7 \; 26 \times pKIM - 1 ( ng / mL ) + 8.7 \; 56 \times CRP ( mg / dl ) - 21.36 \qquad [ Equation 5 ]$$

TABLE 7
Model 6: RNKCS

| | Crude | | | | | |
|---|---|---|---|---|---|---|
| | | | | | (95% | Pseudo |
| Division | Coef. | SE | z | p | CI) | R2 |
| NGAL | 2.15 | 1.027 | 2.095 | 0.0362 | 0.6298 | 0.6828 |
| | | | | | to 4.836 | |
| KIM-1 | 4.178 | 1.646 | 2.538 | 0.0111 | 1.701 | |
| | | | | | to 8.475 | |
| SDMA | 0.4377 | 0.2569 | 1.704 | 0.0885 | 0.04838 | |
| | | | | | to 1.083 | |
| sCr | 1.798 | 2.268 | 0.7927 | 0.4279 | −2.354 | |
| | | | | | to 7.378 | |
| Constant | −21.97 | 8.561 | 2.566 | 0.0103 | −43.63 | |
| | | | | | to −9.004 | |

n = 111,
LR chi2(4) = 42.65,
p < .0001

The estimated function equation from the logistic regression analysis of Model 6 (RNKCS) is as follows:

$$RNKCS = 2.15 \times pNGAL ( ng / mL ) + 4.178 \times pKIM - 1 ( ng / mL ) + 1.7 \; 98 \times sCR ( mg / dl ) + 0.4377 \times SDMA ( μg / dl ) - 21.97 \qquad [ Equation 6 ]$$

Referring to Tables 2 to 7, Model 4 (RNKA), which consists of Age together with pNGAL and pKIM-1, was found to be the optimal model (function) with a high explanatory power (Pseudo R2) of 79.06%.

In the functional equations above, each coefficient (constant) is a value for deriving a model with the highest explanatory power, and does not in itself indicate which variable is more sensitive or important.

Subsequently, any one of the values derived from Equations 1 to 4, which are linear equation models, can be transformed to converge to a value of 1 or 0 by using an exponential function. Here, the prediction equation using the exponential function to classify the risk group of kidney disease (IRIS stage 1, indicating a value of 1) and normal people (indicating a value of 0) is as shown in Equation 7. In Equation 7, the p value is a percentage with a distribution ranging from 0 to 1, wherein a value of less than 0.5 is determined as having no risk of kidney disease, and a value of 0.5 or more is determined as belonging to the risk group of kidney disease.

$$p = exp ( y ) / [ exp ( y ) + 1 ] \qquad [ Equation 7 ]$$

In the equation above, y is a value derived from Equations 1 to 6.

### Experimental Example 2.2. Cutoff Value Ranges for Diagnosing Risk Group of Kidney Disease

To evaluate the accuracy of the optimal model for diagnosing the risk group of kidney disease derived in Section 2-1, the ROC curves were analyzed by using the GraphPad Prism program, and the cutoff values for diagnosing the risk group of kidney disease (normal group vs. risk group and IRIS stages 1 to 4) for NGAL and KIM-1, and RNK, RNKC, RNKS, RNKA, RNKR and RNKCS models derived in Section 2-1 are shown in Tables 8 to 15 below.

TABLE 8
pNGAL

| Cutoff | Sensitivity | 95% | Specificity | 95% | |
|---|---|---|---|---|---|
| value | (%) | CI | (%) | CI | LR+ |
| >1.451 | 99.02 | 94.65% to | 10 | 0.5129% | 1.111 |
| | | 99.95% | | to 40.42% | |
| >3.298 | 81.37 | 72.73% to | 90 | 59.58% | 6.604 |
| | | 87.74% | | to 99.49% | |
| >4.026 | 69.61 | 60.10% to | 100 | 72.25% | — |
| | | 77.69% | | to 100.0% | |

As shown in Table 8, the cutoff value range for the pNGAL concentration to distinguish the risk group of kidney disease may be from 1.451 ng/ml (sensitivity 99.02%, specificity 10%) to 4.026 ng/ml (sensitivity 69.61%, specificity 100%). Preferably, the cutoff value may be 3.30 (sensitivity 81.37%, specificity 90%).

TABLE 9
pKIM-1

| Cutoff | Sensitivity | | Specificity | | |
|---|---|---|---|---|---|
| value | (%) | 95% CI | (%) | 95% CI | LR+ |
| >1.705 | 100 | 96.50% to | 10 | 0.5129% to | 1.286 |
| | | 100.0% | | 40.42% | |
| >3.308 | 66.04 | 56.60% to | 90 | 59.58% to | 8.294 |
| | | 74.35% | | 99.49% | |
| >3.321 | 66.04 | 56.60% to | 100 | 72.25% to | — |
| | | 74.35% | | 100.0% | |

As shown in Table 9, the cutoff value range for the pKIM-1 concentration to distinguish the risk group of kidney disease may be from 1.705 ng/ml (sensitivity 100%, specificity 10%) to 3.321 ng/ml (sensitivity 66.04%, specificity 100%).

TABLE 10
Model 1: RNK

| Cutoff | Sensitivity | | Specificity | | |
|---|---|---|---|---|---|
| value | (%) | 95% CI | (%) | 95% CI | LR |
| >−1.941 | 100 | 96.37% to | 22.22 | 3.948% to | 1.286 |
| | | 100.0% | | 54.74% | |
| >1.408 | 92.16 | 85.28% to | 88.89 | 56.50% to | 8.294 |
| | | 95.97% | | 99.43% | |
| >1.584 | 92.16 | 85.28% to | 100 | 70.09% to | — |
| | | 95.97% | | 100.0% | |

As shown in Table 10, the cutoff value range for the RNK to distinguish the risk group of kidney disease may be from −1.941 (sensitivity 100%, specificity 22.22%) to 1.584 (sensitivity 92.16%, specificity 100.0%).

TABLE 11
Model 2: RNKC

| Cutoff | Sensitivity | | Specificity | | |
|---|---|---|---|---|---|
| value | (%) | 95% CI | (%) | 95% CI | LR |
| >−2.024 | 100 | 96.37% to | 22.22 | 3.948% to | 1.286 |
| | | 100.0% | | 54.74% | |
| >1.257 | 93.14 | 86.51% to | 88.89 | 56.50% to | 8.382 |
| | | 96.64% | | 99.43% | |
| >1.490 | 93.14 | 86.51% to | 100 | 70.09% to | — |
| | | 96.64% | | 100.0% | |

As shown in Table 11, the cutoff value range for the RNKC to distinguish the risk group of kidney disease may be from −2.024 (sensitivity 100%, specificity 22.22%) to 1.490 (sensitivity 93.14%, specificity 100.0%).

TABLE 12
Model 3: RNKS

| Cutoff | Sensitivity | | Specificity | | |
|---|---|---|---|---|---|
| value | (%) | 95% CI | (%) | 95% CI | LR |
| >−3.574 | 100 | 96.37% to | 11.11 | 0.5699% to | 1.125 |
| | | 100.0% | | 43.50% | |
| >1.932 | 93.14 | 86.51% to | 88.89 | 56.50% to | 8.382 |
| | | 96.64% | | 99.43% | |
| >2.085 | 93.14 | 86.51% to | 100 | 70.09% to | — |
| | | 96.64% | | 100.0% | |

As shown in Table 12, the cutoff value range for the RNKS to distinguish the risk group of kidney disease may be from −3.574 (sensitivity 100%, specificity 11.11%) to 2.085 (sensitivity 93.14%, specificity 100.0%).

TABLE 13
Model 4: RNKA

| Cutoff | Sensitivity | | Specificity | | |
|---|---|---|---|---|---|
| value | (%) | 95% CI | (%) | 95% CI | LR |
| >−3.334 | 99.02 | 94.65% to | 0 | 0.000% to | 0.9902 |
| | | 99.95% | | 29.91% | |
| >−2.773 | 99.02 | 94.65% to | 11.11 | 0.5699% to | 1.114 |
| | | 99.95% | | 43.50% | |
| >−0.3581 | 99.02 | 94.65% to | 88.89 | 56.50% to | 8.912 |
| | | 99.95% | | 99.43% | |
| >0.5082 | 99.02 | 94.65% to | 100 | 70.09% to | — |
| | | 99.95% | | 100.0% | |

As shown in Table 13, the cutoff value range for the RNKA to distinguish the risk group of kidney disease may be from −3.334 (sensitivity 100%, specificity 0%) to 0.5082 (sensitivity 99.02%, specificity 100.0%).

TABLE 14
Model 5: RNKR

| Cutoff | Sensitivity | | Specificity | | |
|---|---|---|---|---|---|
| value | (%) | 95% CI | (%) | 95% CI | LR |
| >−3.490 | 100 | 92.73% to | 14.29 | 0.7328% to | 1.167 |
| | | 100.0% | | 51.31% | |
| >−0.1544 | 97.96 | 89.31% to | 85.71 | 48.69% to | 6.857 |
| | | 99.90% | | 99.27% | |
| >−0.07702 | 97.96 | 89.31% to | 100 | 64.57% to | — |
| | | 99.90% | | 100.0% | |

As shown in Table 14, the cutoff value range for the RNKR to distinguish the risk group of kidney disease may be from −3.490 (sensitivity 100%, specificity 14.29%) to −0.07702 (sensitivity 97.96%, specificity 100.0%).

TABLE 15
Model 6: RNKCS

| Cutoff | Sensitivity | | Specificity | | |
|---|---|---|---|---|---|
| value | (%) | 95% CI | (%) | 95% CI | LR |
| >−4.266 | 100 | 96.37% to | 11.11 | 0.5699% to | 1.125 |
| | | 100.0% | | 43.50% | |
| >−2.352 | 99.02 | 94.65% to | 11.11 | 0.5699% to | 1.114 |
| | | 99.95% | | 43.50% | |
| >2.034 | 93.14 | 86.51% to | 88.89 | 56.50% to | 8.382 |
| | | 96.64% | | 99.43% | |
| >2.110 | 93.14 | 86.51% to | 100 | 70.09% to | — |
| | | 96.64% | | 100.0% | |

As shown in Table 15, the cutoff value range for the RNKCS to distinguish the risk group of kidney disease may be from −4.266 (sensitivity 100%, specificity 11.11%) to 2.110 (sensitivity 93.14%, specificity 100.0%).

### 2.3. Evaluation of Diagnostic Accuracy of Derived Optimal Model for Risk Group of Kidney Disease

The ROC curves were analyzed by using the GraphPad Prism program to derive diagnostic accuracy, and from the cutoff value ranges of pNGAL and pKIM-1, and indexes RNK, RNKC, RNKS, RNKA, RNKR, and RNKCS derived in Section 2-2, the optimal cutoff value with the highest value of sensitivity x specificity was derived by a concordance probability method to evaluate the diagnostic value (FIG. 3). In addition, the ranges of diagnostic sensitivity, specificity, accuracy, and reference standard for distinguishing between the normal control and the risk group and IRIS stages 1 to 4 are shown in Table 16 below. According to IRIS 2019, 18<SDMA (μg/dl)<35 or 1.4<sCr (mg/dl)<2.8 indicates Stage 2 CKD, 36<SDMA<54 or 2.9<sCr<5.0 indicates Stage 3 CKD, and 54<SDMA or 5.0<sCr indicates Stage 4 CKD with increased risk of systemic manifestations and uremic crisis.

TABLE 16

| | | | | | Positive | Negative |
|---|---|---|---|---|---|---|
| | Cut-off | AUC | Sensitivity % | Specificity % | likelihood | likelihood |
| Division | (unit) | (95% CI) | (95% CI) | (95% CI) | ratio | ratio |
| sCr | 0.95 (mg/dL) | 0.71* | 50.94 | 90.91 | 5.60 | 0.54 |
| | | (0.59-0.82) | (41.56-60.26) | (62.26-99.53) | | |
| SDMA | 10.50 (μg/dL) | 0.86*** | 83.02 | 77.78 | 3.74 | 0.22 |
| | | (0.76-0.95) | (74.75-88.98) | (45.26-96.05) | | |
| pNGAL | 3.30 (ng/mL) | 0.90*** | 81.37 | 90 | 8.14 | 0.21 |
| | | (0.83-0.97) | (72.73-87.74) | (59.58-99.49) | | |
| pKIM-1 | 3.32 (ng/mL) | 0.90*** | 66.04 | 100 | — | 0.34 |
| | | (0.82-0.99) | (56.6-74.35) | (72.25-100) | | |
| RNK | >1.584 | 0.97*** | 92.16 | 100 | — | 0.08 |
| | | (0.94-1) | (85.28-95.97) | (70.09-100) | | |
| RNKS | >2.085 | 0.98*** | 93.14 | 100 | — | 0.07 |
| | | (0.96-1) | (86.51-96.64) | (70.09-100) | | |
| RNKC | >1.490 | 0.97*** | 93.14 | 100 | — | 0.07 |
| | | (0.94-1) | (86.51-96.64) | (70.09-100) | | |
| RNKA | >0.5082 | 0.99*** | 99.02 | 100 | — | 0.01 |
| | | (0.97-1) | (94.65-99.95) | (70.09-100) | | |
| RNKR | >−0.07702 | 0.98*** | 97.96 | 100 | — | 0.02 |
| | | (0.95-1) | (89.31-99.9) | (64.57-100) | | |
| RNKCS | >2.110 | 0.98*** | 93.14 | 100 | — | 0.07 |
| | | (0.96-1) | (86.51-96.64) | (70.09-100) | | |

Referring FIG. 3 and Table 16, the diagnostic accuracy (area under curve (AUC)) for the risk group or IRIS stages 1 to 4 was high in the order of sCr<SDMA<pNGAL=pKIM-1<RNKC=RNK<RNKS=RNKR=RNKCS<RNKA.

Specifically, an AUC of RNK of the present invention was 0.97 and a value therebelow interpreted as having no kidney disease, i.e., a cutoff value, was 1.584. At the cutoff value, the diagnostic sensitivity was 92.16% and the diagnostic specificity was 100.0%. LR+ (positive likelihood ratio) is incalculable (infinity), and LR− (negative likelihood ratio) is 0.08.

In contrast, the pNGAL had an AUC of 0.90 and a cutoff value of 3.30 ng/ml. At the cutoff value, the diagnostic sensitivity was 81.37% and the diagnostic specificity was 90%. LR+ (positive likelihood ratio) is 8.14, and LR− (negative likelihood ratio) is 0.21.

In addition, the pKIM-1 had an AUC of 0.90 and a cutoff value of 3.32 ng/ml. At the cutoff value, the diagnostic sensitivity was 66.04% % and the diagnostic specificity was 100%. LR+ (positive likelihood ratio) is incalculable (infinity), and LR− (negative likelihood ratio) is 0.34.

The results above indicate that NGAL and pKIM-1 of the present invention can distinguish the risk group of kidney damage or kidney disease with higher accuracy than the conventional index, sCr and SDMA, and also indicate that RNK can distinguish the risk group of kidney disease with significantly higher sensitivity and specificity than pNGAL and pKIM-1.

Additionally, an experiment was conducted to compare clinical efficacy of the sCr, SDMA, pNGAL, and pKIM-1 by baseline stage of disease state. When the diagnostic accuracy was verified as described above, with the disease states including the risk group and IRIS stages 1 to 4 (FIG. 3), it was found that the AUC, sensitivity, and specificity between pKIM-1 and pNGAL were generally similar, but were higher than those of SDMA and sCr.

When the disease state is at IRIS stage 1 or higher, the diagnostic sensitivity, specificity, accuracy, and reference range are shown in Table 17 below.

TABLE 17

| | | | | | Positive | Negative |
|---|---|---|---|---|---|---|
| | Cut-off | AUC | Sensitivity % | Specificity % | likelihood | likelihood |
| Division | (unit) | (95% CI) | (95% CI) | (95% CI) | ratio | ratio |
| sCr | 0.95 | 0.65* | 53.26 | 76 | 2.22 | 0.62 |
| | (mg/dL) | (0.55-0.75) | (43.14-63.12) | (56.57-88.5) | | |
| SDMA | 13.50 | 0.84*** | 70.65 | 91.3 | 8.12 | 0.32 |
| | (μg/dL) | (0.77-0.92) | (60.67-78.98) | (73.2-98.45) | | |
| pNGAL | 4.19 | 0.88*** | 76.14 | 87.5 | 6.09 | 0.27 |
| | (ng/mL) | (0.8-0.95) | (66.26-83.83) | (69-95.66) | | |
| pKIM-1 | 3.70 | 0.72*** | 57.61 | 79.17 | 2.77 | 0.54 |
| | (ng/mL) | (0.62-0.82) | (47.41-67.2) | (59.53-90.76) | | |

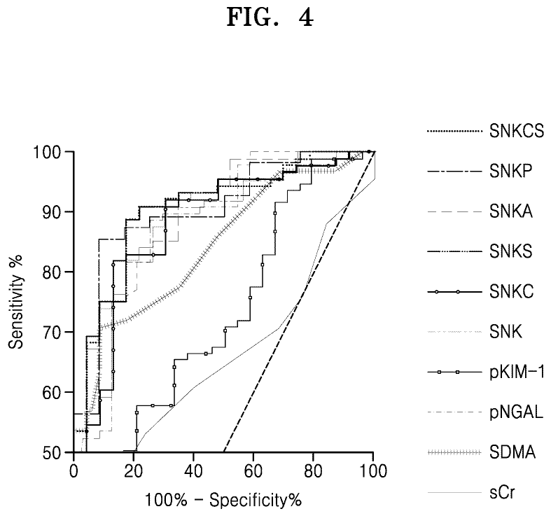

As shown in FIG. 4, the AUC of pNGAL was higher than that of SDMA, and the AUC of pKIM-1 was higher than that of sCr.

In addition, when the disease state is at IRIS stage 2 or higher, the diagnostic sensitivity, specificity, accuracy, and reference range are shown in Table 18 below.

TABLE 18

| | | | | | Positive | Negative |
|---|---|---|---|---|---|---|
| | Cut-off | AUC | Sensitivity % | Specificity % | likelihood | likelihood |
| Division | (unit) | (95% CI) | (95% CI) | (95% CI) | ratio | ratio |
| sCr | 1.25 | 0.89*** | 69.39 | 95.59 | 15.73 | 0.32 |
| | (mg/dL) | (0.83-0.96) | (55.47-80.48) | (87.81-98.8) | | |
| SDMA | 16.50 | 0.95*** | 85.71 | 98.48 | 56.39 | 0.15 |
| | (μg/dL) | (0.9-0.99) | (73.33-92.9) | (91.9-99.92) | | |
| pNGAL | 4.89 | 0.75*** | 82.98 | 60 | 2.07 | 0.28 |
| | (ng/mL) | (0.66-0.84) | (69.86-91.11) | (47.86-71.03) | | |
| pKIM-1 | 4.14 | 0.88*** | 79.59 | 86.57 | 5.93 | 0.24 |
| | (ng/mL) | (0.81-0.95) | (66.36-88.52) | (76.4-92.77) | | |

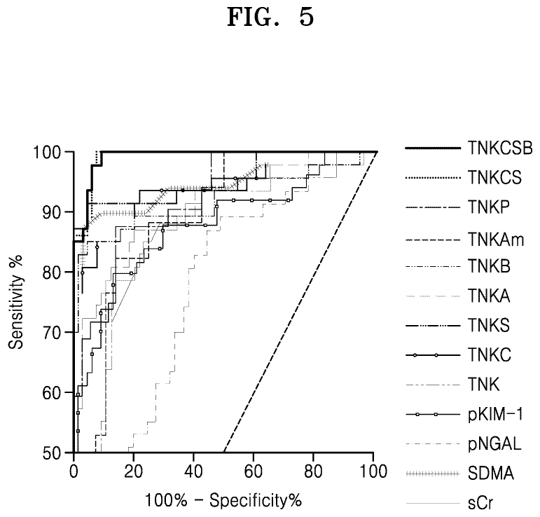

As shown in FIG. 5, since sCr and SDMA were used as standards for IRIS stages, the accuracy of SDMA and sCr was found to be higher than that of other biomarkers.

### Experimental Example 3. Optimal Model for Diagnosing Stage 1 Kidney Disease

### Experimental Example 3.1. Derivation of Diagnostic Model for Kidney Disease at Stage 1-Distinguishing of Normal Group and Risk Group Vs. IRIS Stages 1 to 4

Optimal indexes for distinguishing of Stage 1 group of kidney disease (normal group and risk group vs. IRIS stages 1 to 4) were to be derived. Specifically, risk factors associated with kidney disease (including chemical test results or clinical indexes) were set as multiple independent variables, and the prevalence (likelihood of development) at IRIS stages 1 to 4 was set as a dependent variable. Then, by crude logistic regression analysis of the Graphpad Prism program, the extent of influence of the multiple independent variables on the dependent variable was confirmed and only the variables that were significant at a significance level of 0.05 were combined (Table 19).

TABLE 19

| | Crude logistic regression analysis | | | | | | |
|---|---|---|---|---|---|---|---|
| | | | | | | (95% | Pseudo |
| Division | n | OR | SE | z | p | CI) | R2 |
| NGAL | 112 | 1.85 | 0.6343 | 3.766 | 0.0002 | 1.396~2.66 | 0.3166 |
| KIM-1 | 116 | 1.902 | 0.7839 | 2.846 | 0.0044 | 1.297~3.145 | 0.1071 |
| SDMA | 115 | 1.407 | 1.065 | 3.712 | 0.0002 | 1.201~1.724 | 0.2534 |
| sCr | 117 | 3.907 | 0.5456 | 2.378 | 0.0174 | 1.53~14.45 | 0.06252 |
| Age | 117 | 1.299 | 0.6021 | 4.087 | <0.0001 | 1.154~1.487 | 0.1815 |
| BW | 116 | 0.7859 | 0.7002 | 1.719 | 0.0856 | 0.5937~1.034 | 0.0235 |
| BP | 105 | 1.014 | 2.147 | 0.879 | 0.3794 | 0.9832~1.047 | 0.00673 |
| BUN | 117 | 1.073 | 0.5053 | 2.8 | 0.0051 | 1.028~1.135 | 0.0911 |
| BCR | 117 | 1.03 | 0.5015 | 1.421 | 0.1553 | 0.9938~1.077 | 0.01569 |
| Globulin | 103 | 2.4 | 1.666 | 1.898 | 0.0577 | 1.021~6.328 | 0.03462 |
| Amylase | 45 | 1.006 | 1.619 | 2.118 | 0.0342 | 1.002~1.013 | 0.1832 |
| CRP | 57 | 6.987 | 0.5504 | 1.867 | 0.0619 | 1.489~86.59 | 0.1697 |
| Phosphorus | 68 | 3.674 | 1.501 | 3.014 | 0.0026 | 1.738~9.623 | 0.2339 |
| In organic | | | | | | | |

Crude: Univariate logistic

The functional relation between the above-mentioned combined independent and dependent variables was modeled through multiple logistic regression analysis of the Graphpad Prism program, and the Equation was inferred. Here, the logistic regression analysis is a binary algorithm for modeling the relation between data on risk factors and the likelihood of developing kidney disease.

By such modeling, SNK, SNKC, SNKS, SNKA, SNKP and SNKCS as the optimal models (functions) with the highest pseudo R2 (referring to explanatory power, the intensity of the relation between the dependent and independent variables) were derived (Tables 20 to 23). For reference, SNK is a function using pNGAL and pKIM-1, SNKC is a function using pNGAL, pKIM-1, and sCr, SNKS is a function using pNGAL, pKIM-1, and SMDA, SNKA is a function using pNGAL, pKIM-1, and age, SNKR is a function using pNGAL, pKIM-1, and inorganic phosphorus, and SNKCS is a function using pNGAL, pKIM-1, sCr, and SDMA.

TABLE 20
Model: SNK

| | Crude | | | | | |
|---|---|---|---|---|---|---|
| | | | | | (95% | Pseudo |
| Division | Coef. | SE | z | p | CI) | R2 |
| NGAL (ng/mL) | 0.5541 | 0.165 | 3.357 | 0.0008 | 0.2690 | 0.3213 |
| | | | | | to 0.9201 | |
| KIM-1 (ng/mL) | 0.3766 | 0.2574 | 1.463 | 0.1435 | −0.05742 | |
| | | | | | to 0.9412 | |
| Constant | −2.614 | 1.046 | 2.5 | 0.0124 | −4.866 | |
| | | | | | to −0.7600 | |

n = 111,
LR chi2(2) = 36.40,
p < .0001

In the top entry of the table, Coef. represents a regression coefficient indicating the magnitude of the influence of the independent variables on the dependent variable, S.E. represents a standard error, 95% CI (confidence interval) represents the lower and upper limits of the 95% confidence interval of the estimated coefficient, z refers to a t-distribution statistic obtained by dividing the estimated coefficient by the S.E., p refers to a test statistic for the extent to which the null hypothesis can be rejected, and Pseudo R2 refers to the extent to which the dependent variable is explained by the independent variables, i.e., the explanatory power (predictive power).

The estimated function equation from the logistic regression analysis of Model 7 (SNK) is as follows:

$$SNK ( y ) = 0.5541 \times pNGAL ( ng / mL ) + 0.37 \; 66 \times pKIM - 1 ( ng / mL ) - 2.614 \qquad [ Equation 8 ]$$

TABLE 21
Model 8: SNKC

| | Crude | | | | | |
|---|---|---|---|---|---|---|
| | | | | | (95% | Pseudo |
| Division | Coef. | SE | z | p | CI) | R2 |
| NGAL | 0.5522 | 0.1657 | 3.333 | 0.0009 | 0.2659 | 0.3244 |
| (ng/mL) | | | | | to 0.9198 | |
| KIM-1 | 0.3146 | 0.2764 | 1.138 | 0.255 | −0.1517 | |
| (ng/mL) | | | | | to 0.9128 | |
| Creatinine | 0.4417 | 0.7777 | 0.5679 | 0.5701 | −0.9532 | |
| | | | | | to 2.160 | |
| Constant | −2.792 | 1.118 | 2.497 | 0.0125 | −5.265 | |
| | | | | | to −0.8363 | |

n = 111,
LR chi2(3) = 36.74,
p < .0001

The estimated function equation from the logistic regression analysis of Model 8 (SNKC) is as follows:

$$SNKC ( y ) = 0.5522 \times pNGAL ( ng / mL ) + 0.31 \; 46 \times pKIM - 1 ( ng / mL ) + 0.4417 \times sCr ( mg / dl ) - 2.792 \qquad [ Equation 9 ]$$

TABLE 22
Model 9: SNKS

| | Crude | | | | | |
|---|---|---|---|---|---|---|
| | | | | | (95% | Pseudo |
| Division | Coef. | SE | z | p | CI) | R2 |
| NGAL | 0.442 | 0.1601 | 2.761 | 0.0058 | 0.1677 | 0.4048 |
| (ng/mL) | | | | | to 0.7995 | |
| KIM-1 | 0.001992 | 0.3291 | 0.006052 | 0.9952 | −0.6138 | |
| (ng/mL) | | | | | to 0.6873 | |
| SDMA | 0.2562 | 0.09982 | 2.567 | 0.0103 | 0.08261 | |
| | | | | | to 0.4765 | |
| Constant | −4.079 | 1.365 | 2.988 | 0.0028 | −7.127 | |
| | | | | | to −1.722 | |

n = 111,
LR chi2(3) = 45.85,
p < .0001

The estimated function equation from the logistic regression analysis of Model 9 (SNKS) is as follows:

$$SNKS ( y ) = 0.442 \times pNGAL ( ng / mL ) + 0.0019 \; 92 \times pKIM - 1 ( ng / mL ) + 0.256 \times SDMA ( μg / dl ) - 4.079 \qquad [ Equation 10 ]$$

TABLE 23
Model 10: SNKA

| | Crude | | | | | |
|---|---|---|---|---|---|---|
| | | | | | (95% | Pseudo |
| Division | Coef. | SE | z | p | CI) | R2 |
| NGAL | 0.447 | 0.1699 | 2.632 | 0.0085 | 0.1621 | 0.3807 |
| (ng/mL) | | | | | to 0.8222 | |
| KIM-1 | 0.2079 | 0.2395 | 0.8679 | 0.3854 | −0.1550 | |
| (ng/mL) | | | | | to 0.7786 | |
| Age | 0.2108 | 0.08657 | 2.436 | 0.0149 | 0.04981 | |
| | | | | | to 0.3949 | |
| Constant | −3.55 | 1.136 | 3.125 | 0.0018 | −6.092 | |
| | | | | | to −1.557 | |

n = 111,
LR chi2(3) = 43.12,
p < .0001

The estimated function equation from the logistic regression analysis of Model 10 (SNKA) is as follows:

$$SNKA ( y ) = 0.447 \times pNGAL ( ng / mL ) + 0.20 \; 79 \times pKIM - 1 ( ng / mL ) + 0.2108 \times Age ( year ) - 3.55 \qquad [ Equation 11 ]$$

TABLE 24
Model 11: SNKP

| | Crude | | | | | |
|---|---|---|---|---|---|---|
| | | | | | (95% | Pseudo |
| Division | Coef. | SE | z | p | CI) | R2 |
| NGAL | 0.4599 | 0.2185 | 2.104 | 0.0353 | 0.09859 | 0.4172 |
| (ng/mL) | | | | | to 0.9728 | |
| KIM-1 | 0.3363 | 0.3761 | 0.8942 | 0.3712 | −0.3390 | |
| (ng/mL) | | | | | to 1.153 | |
| Inorganic | 1.004 | 0.4893 | 2.052 | 0.0402 | 0.1271 | |
| phosphorus | | | | | to 2.095 | |
| Constant | −5.678 | 2.263 | 2.509 | 0.0121 | −10.93 | |
| | | | | | to −1.792 | |

n = 67,
LR chi2(3) = 2628,
p < .0001

The estimated function equation from the logistic regression analysis of Model 11 (SNKP) is as follows:

$$SNKP ( y ) = 0.4599 \times pNGAL ( ng / mL ) + 0.33 \; 63 \times pKIM - 1 ( ng / mL ) + 1.004 \times Inorganic phosphorus ( mg / dl ) - 5.678 \qquad [ Equation 12 ]$$

TABLE 25
Model 12: SNKCS

| | Crude | | | | | |
|---|---|---|---|---|---|---|
| | | | | | (95% | Pseudo |
| Division | Coef. | SE | z | p | CI) | R2 |
| NGAL | 0.4406 | 0.1608 | 2.74 | 0.0062 | 0.1656 | 0.4048 |
| (ng/mL) | | | | | to 0.8000 | |
| KIM-1 | 0.007931 | 0.3372 | 0.02352 | 0.9812 | −0.6201 | |
| (ng/mL) | | | | | to 0.7089 | |
| SDMA | 0.258 | 0.1022 | 2.525 | 0.0116 | 0.08021 | |
| | | | | | to 0.4826 | |
| Creatinine | −0.07766 | 0.9396 | 0.08265 | 0.9341 | −1.865 | |
| | | | | | to 1.861 | |
| Constant | −4.048 | 1.41 | 2.87 | 0.0041 | −7.246 | |
| | | | | | to −1.644 | |

n = 111,
LR chi2(4) = 45.86,
p < .0001

The estimated function equation from the logistic regression analysis of Model 12 (SNKCS) is as follows:

$$SNKCS ( y ) = 0.4406 \times pNGAL ( ng / mL ) + 0.007931 \times pKIM - 1 ( ng / mL ) - 0.07766 \times sCr ( mg / dl ) + 0.258 \times SDMA ( μg / dl ) - 4.048 \qquad [ Equation 13 ]$$

Referring to Tables 20 to 25, Model 11 (SNKP), which combines inorganic phosphorus with pNGAL, pKIM-1, and the like was found to be the optimal model (function) with the highest explanatory power (Pseudo R2) of 41.72%.

Subsequently, any one of the values derived from Equations 8 to 13, which are linear equation models, can be transformed to converge to a value of 1 or 0 by using an exponential function. Here, the prediction equation using the exponential function to classify the Stage 1 kidney disease (IRIS stages 1 to 4, indicating a value of 1) and normal people and the risk group of kidney disease (indicating a value of 0) is as shown in Equation 14. In Equation 14, the p value is a percentage with a distribution ranging from 0 to 1, wherein a value of less than 0.5 is determined as having no kidney disease, and a value of 0.5 or more is determined as being at Stage 1 kidney disease.

$$p = exp ( y ) / [ exp ( y ) + 1 ] \qquad [ Equation 14 ]$$

In the equation above, y is a value derived from Equations 1 to 6.

### Experimental Example 3.2. Cutoff Value Ranges for Diagnosing Stage 1 Kidney Disease

To evaluate the accuracy of the optimal model for diagnosing Stage 1 kidney disease derived in Experimental Example 3.1, the ROC curves were analyzed by using the GraphPad Prism program, and the cutoff values for diagnosing Stage 1 kidney disease (normal group, risk group vs. IRIS stages 1 to 4) for NGAL and KIM-1, and SNK, SNKC, SNKS, SNKA, SNKP and SNKCS models derived in Section 3-1 are shown in Tables 26 to 33 below.

TABLE 26
pNGAL

| Cutoff | Sensitivity | | Specificity | | |
|---|---|---|---|---|---|
| value | (%) | 95% CI | (%) | 95% CI | LR+ |
| >1.932 | 100 | 95.82% to | 41.67 | 24.47% to | 1.714 |
| | | 100.0% | | 61.17% | |
| >4.192 | 76.14 | 66.26% to | 87.5 | 69.00% to | 6.091 |
| | | 83.83% | | 95.66% | |
| >9.722 | 34.09 | 25.04% to | 100 | 86.20% to | — |
| | | 44.47% | | 100.0% | |

As shown in Table 26, the cutoff value range for the pNGAL concentration to distinguish Stage 1 kidney disease may be from 1.932 ng/ml (sensitivity 100%, specificity 41.67%) to 9.722 ng/ml (sensitivity 34.09%, specificity 100%).

TABLE 27
pKIM-1

| Cutoff | Sensitivity | | Specificity | | |
|---|---|---|---|---|---|
| value | (%) | 95% CI | (%) | 95% CI | LR+ |
| >1.705 | 100 | 95.99% to | 4.167 | 0.2137% to | 1.043 |
| | | 100.0% | | 20.24% | |
| >3.701 | 57.61 | 47.41% to | 79.17 | 59.53% to | 2.765 |
| | | 67.20% | | 90.76% | |
| >5.138 | 33.7 | 24.86% to | 100 | 86.20% to | — |
| | | 43.83% | | 100.0% | |

As shown in Table 27, the cutoff value range for the pKIM-1 concentration to distinguish Stage 1 kidney disease may be from 1.705 ng/ml (sensitivity 100%, specificity 4.167%) to 5.138 ng/ml (sensitivity 33.7%, specificity 100.0%).

TABLE 28
Model: SNK

| Cutoff | Sensitivity | | Specificity | | |
|---|---|---|---|---|---|
| value | (%) | 95% CI | (%) | 95% CI | LR |
| >−0.7360 | 100 | 95.82% to | 8.696 | 1.545% to | 1.095 |
| | | 100.0% | | 26.80% | |
| >0.9713 | 80.68 | 71.22% to | 86.96 | 67.87% to | 6.186 |
| | | 87.57% | | 95.46% | |
| >4.229 | 36.36 | 27.08% to | 100 | 85.69% to | — |
| | | 46.79% | | 100.0% | |

As shown in Table 28, the cutoff value range for the SNK to distinguish Stage 1 kidney disease may be from −0.7360 (sensitivity 100%, specificity 8.696%) to 4.229 (sensitivity 36.36%, specificity 100.0%).

TABLE 29
Model 8: SNKC

| Cutoff | Sensitivity | | Specificity | | |
|---|---|---|---|---|---|
| value | (%) | 95% CI | (%) | 95% CI | LR |
| >−0.6980 | 100 | 95.82% to | 8.696 | 1.545% to | 1.095 |
| | | 100.0% | | 26.80% | |
| >0.8345 | 81.82 | 72.49% to | 86.96 | 67.87% to | 6.273 |
| | | 88.49% | | 95.46% | |
| >4.256 | 35.23 | 26.06% to | 100 | 85.69% to | — |
| | | 45.63% | | 100.0% | |

As shown in Table 29, the cutoff value range for the SNK to distinguish Stage 1 kidney disease may be from −0.6980 (sensitivity 100%, specificity 8.696%) to 4.256 (sensitivity 35.23%, specificity 100.0%).

TABLE 30
Model 9: SNKS

| Cutoff | Sensitivity | | Specificity | | |
|---|---|---|---|---|---|
| value | (%) | 95% CI | (%) | 95% CI | LR |
| >−1.246 | 100 | 95.82% to | 21.74 | 9.664% to | 1.278 |
| | | 100.0% | | 41.90% | |
| >0.4947 | 88.64 | 80.33% to | 82.61 | 62.86% to | 5.097 |
| | | 93.71% | | 93.02% | |
| >3.307 | 53.41 | 43.06% to | 100 | 85.69% to | — |
| | | 63.47% | | 100.0% | |

As shown in Table 30, the cutoff value range for the SNKS to distinguish Stage 1 kidney disease may be from −1.246 (sensitivity 100%, specificity 21.74%) to 3.307 (sensitivity 53.41%, specificity 100.0%).

TABLE 31
Model 10: SNKA

| Cutoff | Sensitivity | | Specificity | | |
|---|---|---|---|---|---|
| value | (%) | 95% CI | (%) | 95% CI | LR |
| >−1.667 | 100 | 95.82% to | 13.04 | 4.538% to | 1.15 |
| | | 100.0% | | 32.13% | |
| >0.9648 | 81.82 | 72.49% to | 82.61 | 62.86% to | 4.705 |
| | | 88.49% | | 93.02% | |
| >4.106 | 39.77 | 30.18% to | 100 | 85.69% to | — |
| | | 50.22% | | 100.0% | |

As shown in Table 31, the cutoff value range for the SNKA to distinguish Stage 1 kidney disease may be from −1.667 (sensitivity 100%, specificity 13.04%) to 4.106 (sensitivity 39.77%, specificity 100.0%).

TABLE 32
Model 11: SNKP

| Cutoff | Sensitivity | | Specificity | | |
|---|---|---|---|---|---|
| value | (%) | 95% CI | (%) | 95% CI | LR |
| >−0.7626 | 100 | 93.47% to | 25 | 8.894% to | 1.333 |
| | | 100.0% | | 53.23% | |
| >0.7632 | 85.45 | 73.84% to | 91.67 | 64.61% to | 10.25 |
| | | 92.44% | | 99.57% | |
| >3.650 | 56.36 | 43.27% to | 100 | 75.75% to | — |
| | | 68.63% | | 100.0% | |

As shown in Table 32, the cutoff value range for the SNKP to distinguish Stage 1 kidney disease may be from −0.7626 (sensitivity 100%, specificity 25%) to 3.650 (sensitivity 56.36%, specificity 100.0%).

TABLE 33
Model 12: SNKCS

| Cutoff | Sensitivity | 95% | Specificity | 95% | |
|---|---|---|---|---|---|
| value | (%) | CI | (%) | CI | LR |
| >−1.258 | 100 | 95.82% to | 21.74 | 9.664% to | 1.278 |
| | | 100.0% | | 41.90% | |
| >0.4832 | 88.64 | 80.33% to | 82.61 | 62.86% to | 5.097 |
| | | 93.71% | | 93.02% | |
| >3.298 | 53.41 | 43.06% to | 100 | 85.69% to | — |
| | | 63.47% | | 100.0% | |

As shown in Table 33, the cutoff value range for the SNKCS to distinguish Stage 1 kidney disease may be from −1.258 (sensitivity 100%, specificity 21.74%) to 3.298 (sensitivity 53.41%, specificity 100.0%).

### Experimental Example 3.3. Evaluation of Diagnostic Accuracy of Derived Optimal Model for Stage 1 Kidney Disease

The ROC curves were analyzed by using the GraphPad Prism program, and from the cutoff value ranges for NGAL, KIM-1, SNK, SNKC, SNKS, SNKA, SNKP and SNKCS derived in Experimental Example 3.2, the optimal cutoff value with the highest diagnostic accuracy was derived (FIG. 4). In addition, the ranges of diagnostic sensitivity, specificity, accuracy, and reference standard for distinguishing between the normal control and the risk group and Stage 1 CKD (IRIS stages 1 to 4) are shown in Table 34 below.

TABLE 34

| | | | | | Positive | Negative |
|---|---|---|---|---|---|---|
| | Cut-off | AUC | Sensitivity % | Specificity % | likelihood | likelihood |
| Division | (unit) | (95% CI) | (95% CI) | (95% CI) | ratio | ratio |
| sCr | 0.95 | 0.65* | 53.26 | 76 | 2.22 | 0.62 |
| | (mg/dL) | (0.55-0.75) | (43.14-63.12) | (56.57-88.5) | | |
| SDMA | 13.50 | 0.84*** | 70.65 | 91.3 | 8.12 | 0.32 |
| | (μg/dL) | (0.77-0.92) | (60.67-78.98) | (73.2-98.45) | | |
| pNGAL | 4.19 | 0.88*** | 76.14 | 87.5 | 6.09 | 0.27 |
| | (ng/mL) | (0.8-0.95) | (66.26-83.83) | (69-95.66) | | |
| pKIM-1 | 3.70 | 0.72*** | 57.61 | 79.17 | 2.77 | 0.54 |
| | (ng/mL) | (0.62-0.82) | (47.41-67.2) | (59.53-90.76) | | |
| SNK | >0.8887 | 0.87*** | 81.82 | 86.96 | 6.27 | 0.21 |
| | | (0.8-0.95) | (72.49-88.49) | (67.87-95.46) | | |
| SNKS | >0.4947 | 0.91*** | 88.64 | 82.61 | 5.10 | 0.14 |
| | | (0.85-0.97) | (80.33-93.71) | (62.86-93.02) | | |
| SNKC | >0.8345 | 0.87*** | 81.82 | 86.96 | 6.27 | 0.21 |
| | | (0.8-0.95) | (72.49-88.49) | (67.87-95.46) | | |
| SNKA | >0.9648 | 0.89*** | 81.82 | 82.61 | 4.71 | 0.22 |
| | | (0.83-0.96) | (72.49-88.49) | (62.86-93.02) | | |
| SNKP | >0.7632 | 0.9*** | 85.45 | 91.67 | 10.26 | 0.16 |
| | | (0.82-0.99) | (73.84-92.44) | (64.61-99.57) | | |
| SNKCS | >0.4832 | 0.91*** | 88.64 | 82.61 | 5.10 | 0.14 |
| | | (0.85-0.97) | (80.33-93.71) | (62.86-93.02) | | |

Referring to FIG. 4 and Table 34, the diagnostic accuracy (AUC) for IRIS stages 1 to 4 was high in the order of sCr <pKIM-1<SDMA<SNK=SNKC<pNGAL<SNKA<SNKP<SNKS=SNKCS.

Specifically, an AUC of SNK of the present invention was 0.87 and a value therebelow interpreted as not being at Stage 1 kidney disease, i.e., a cutoff value, was 0.8887. At the cutoff value, the diagnostic sensitivity was 81.82% and the diagnostic specificity was 86.96%. LR+ (positive likelihood ratio) is 6.27, and LR− (negative likelihood ratio) is 0.21.

In addition, an AUC of SNKS of the present invention was 0.91 and a value therebelow interpreted as not being at Stage 1 kidney disease, i.e., a cutoff value, was 0.4947. At the cutoff value, the diagnostic sensitivity was 88.64% and the diagnostic specificity was 82.61%. LR+ (positive likelihood ratio) is 5.10, and LR− (negative likelihood ratio) is 0.14.

In addition, an AUC of SNKCS of the present invention was 0.91 and a value therebelow interpreted as not being at Stage 1 kidney disease, i.e., a cutoff value, was 0.4832. At the cutoff value, the diagnostic sensitivity was 88.64% and the diagnostic specificity was 82.61%. LR+ (positive likelihood ratio) is 5.10, and LR− (negative likelihood ratio) is 0.14.

In contrast, the pNGAL had an AUC of 0.88 and a cutoff value of 4.19 ng/mL. At the cutoff value, the diagnostic sensitivity was 76.14% and the diagnostic specificity was 87.5%. LR+ (positive likelihood ratio) is 6.09, and LR− (negative likelihood ratio) is 0.27.

In addition, the pKIM-1 had an AUC of 0.72 and a cutoff value of 3.70 ng/ml. At the cutoff value, the diagnostic sensitivity was 57.61% and the diagnostic specificity was 79.17%. LR+ (positive likelihood ratio) is 2.77, and LR− (negative likelihood ratio) is 0.54.

The results above indicate that NGAL, SNK, SNKC, SNKS, SNKA, SNKP and SNKCS of the present invention can distinguish Stage 1 kidney disease with higher accuracy, sensitivity and specificity than sCr and SDMA. That is, the indexes of the present invention are found to enable diagnosis of kidney disease at a level equivalent to or higher than the conventional indexes.

### Experimental Example 4. Optimal Model for Diagnosing Stage 2 Kidney Disease

### Experimental Example 4.1. Derivation of Diagnostic Model for Kidney Disease Group-Distinguishing Between Normal Group, Risk Group, and IRIS Stage 1 vs IRIS Stages 2 to 4

Optimal indexes for distinguishing of kidney disease groups (normal group and IRIS stage 1 vs. IRIS stages 2 to 4) were to be derived. Specifically, risk factors associated with kidney disease (including chemical test results or clinical indexes) were set as multiple independent variables, and the prevalence (likelihood of development) of kidney disease (IRIS stages 2 to 4) was set as a dependent variable. Then, by simple logistic regression analysis of the Graphpad Prism program, the extent of influence of the multiple independent variables on the dependent variable was confirmed and only the variables that were significant at a significance level of 0.05 were combined (Table 35). As a result of the crude analysis, the dependent variables were correlated with the independent variables including NGAL, KIM-1, SDMA, SDMA, sCr, Age, amylase, inorganic phosphorus and BUN, and thus the optimal combination was set using these nine variables.

TABLE 35

| | Crude Logistic regression analysis | | | | | | |
|---|---|---|---|---|---|---|---|
| | | | | | | (95% | Pseudo |
| Division | n | OR | SE | z | p | CI) | R2 |
| NGAL | 112 | 1.111 | 0.325 | 3.162 | 0.0016 | 1.048~1.195 | 0.1389 |
| KIM-1 | 116 | 3.526 | 1.024 | 5.087 | <0.0001 | 2.283~6.058 | 0.4864 |
| SDMA | 115 | 1.768 | 1.893 | 4.709 | <0.0001 | 1.447~2.347 | 0.7042 |
| sCr | 117 | 101.1 | 0.9679 | 4.872 | <0.0001 | 19.65~832.9 | 0.5204 |
| Age | 117 | 1.246 | 0.6729 | 3.877 | 0.0001 | 1.123~1.405 | 0.164 |
| BW | 116 | 1 | 0.5547 | 1.024E−05 | >0.9999 | 0.7899~1.263 | 9.34E−13 |
| BP | 105 | 1.005 | 1.703 | 0.3873 | 0.6986 | 0.9805~1.03 | 0.001423 |
| BUN | 117 | 1.127 | 0.6066 | 5.027 | <0.0001 | 1.081~1.188 | 0.4261 |
| BCR | 117 | 1.01 | 0.3652 | 0.8103 | 0.4178 | 0.9856~1.037 | 0.005945 |
| Globulin | 103 | 2.067 | 1.375 | 2.012 | 0.0442 | 1.042~4.346 | 0.04186 |
| Amylase | 45 | 1.003 | 0.9546 | 2.63 | 0.0085 | 1.001~1.006 | 0.306 |
| CRP | 57 | 1.786 | 0.3827 | 2.196 | 0.0281 | 1.224~3.495 | 0.2335 |
| Phosphorus | 68 | 2.035 | 1.043 | 3.018 | 0.0025 | 1.37~3.437 | 0.2272 |
| In organic | | | | | | | |

Crude : Univariate logistic

The functional relation between the combined independent and dependent variables was modeled through multiple logistic regression analysis of the Graphpad Prism program to deduce an equation. Here, the logistic regression analysis is a binary algorithm for modeling the relation between data on risk factors and the likelihood of developing kidney disease.

By such modeling, TNK, TNKC, TNKS, TNKA, TNKP, TNKAm, TNKCS, TNKB and TNKCSB as the optimal models (functions) with the highest pseudo R2 (referring to explanatory power, the intensity of the relation between the dependent and independent variables) were derived (Tables 36 to 44). For reference, TNK is a function using pNGAL and pKIM-1, TNKC is a function using pNGAL, pKIM-1, and sCr, TNKS is a function using pNGAL, pKIM-1, and SDMA, TNKA is a function using pNGAL, pKIM-1, and age, TNKP is a function using pNGAL, pKIM-1, and Inorganic phosphorus, TNKAm is a function using pNGAL, pKIM-1, and Amylase, TNKCS is a function using pNGAL, pKIM-1, sCr, and SDMA, TNKB is a function using pNGAL, pKIM-1, and BUN, and TNKCSB is a function using pNGAL, pKIM-1, sCr, SDMA, and BUN.

TABLE 36
Model 13: TNK

| | Crude | | | | | |
|---|---|---|---|---|---|---|
| | | | | | (95% | Pseudo |
| Division | Coef. | SE | z | p | CI) | R2 |
| NGAL | 0.03031 | 0.04578 | 0.6621 | 0.5079 | −0.06524 | 0.4078 |
| (ng/mL) | | | | | to 0.1148 | |
| KIM-1 | 1.187 | 0.2489 | 4.769 | <0.0001 | 0.7496 | |
| (ng/mL) | | | | | to 1.731 | |
| Constant | −5.538 | 1.037 | 5.338 | <0.0001 | −7.805 | |
| | | | | | to −3.708 | |

n = 111,
LR chi2(2) = 61.68,
p < .0001

In the top entry of the table, Coef. represents a regression coefficient indicating the magnitude of the influence of the independent variables on the dependent variable, S.E. represents a standard error, 95% CI (confidence interval) represents the lower and upper limits of the 95% confidence interval of the estimated coefficient, z refers to a t-distribution statistic obtained by dividing the estimated coefficient by the S.E., p refers to a test statistic for the extent to which the null hypothesis can be rejected, and Pseudo R2 refers to the extent to which the dependent variable is explained by the independent variables, i.e., the explanatory power (predictive power).

The estimated function equation from the logistic regression analysis of Model 13 (TNK) is as follows:

$$TNK ( y ) = 0.03031 \times pNGAL ( ng / mL ) + 1.187 \times pKIM - 1 ( ng / mL ) - 5.538 \qquad [ Equation 15 ]$$

TABLE 37
Model 14: TNKC

| | Crude | | | | | |
|---|---|---|---|---|---|---|
| | | | | | (95% | Pseudo |
| Division | Coef. | SE | z | p | CI) | R2 |
| NGAL | −0.01621 | 0.06481 | 0.2501 | 0.8025 | −0.1380 | 0.5789 |
| (ng/mL) | | | | | to 0.1067 | |
| KIM-1 | 0.9737 | 0.2869 | 3.393 | 0.0007 | 0.4760 | |
| (ng/mL) | | | | | to 1.608 | |
| Creatinine | 3.773 | 1.03 | 3.662 | 0.0003 | 2.014 | |
| | | | | | to 6.096 | |
| Constant | −8.309 | 1.596 | 5.207 | <0.0001 | −11.97 | |
| | | | | | to −5.607 | |

n = 111,
LR chi2(3) = 87.56,
p < .0001

The estimated function equation from the logistic regression analysis of Model 14 (TNKC) is as follows:

$$TNKC ( y ) = - 0.01621 \times pNGAL ( ng / mL ) + 0.9 \; 737 \times pKIM - 1 ( \text{ng/mL} ) + 3.773 \times sCr ( mg / dl ) - 8.309 \qquad [ Equation 16 ]$$

TABLE 38
Model 15: TNKS

| | Crude | | | | | |
|---|---|---|---|---|---|---|
| | | | | | (95% | Pseudo |
| Division | Coef. | SE | z | p | CI) | R2 |
| NGAL | −0.01627 | 0.0648 | 0.2511 | 0.8017 | −0.1550 | 0.6532 |
| (ng/mL) | | | | | to 0.09814 | |
| KIM-1 | 0.631 | 0.3246 | 1.944 | 0.0519 | 0.04888 | |
| (ng/mL) | | | | | to 1.327 | |
| SDMA | 0.4914 | 0.1284 | 3.827 | 0.0001 | 0.2780 | |
| | | | | | to 0.7913 | |
| Constant | −10.55 | 2.253 | 4.685 | <0.0001 | −15.91 | |
| | | | | | to −6.873 | |

n = 111,
LR chi2(3) = 98.80,
p < .0001

The estimated function equation from the logistic regression analysis of Model 15 (TNKS) is as follows:

$$TNKS ( y ) = - 0.01627 \times pNGAL ( ng / mL ) + 0.6 \; 31 \times pKIM - 1 ( ng / mL ) + 0.4914 \times sCr ( mg / dl ) - 10.55 \qquad [ Equation 17 ]$$

TABLE 39
Model 16: TNKA

| | Crude | | | | | |
|---|---|---|---|---|---|---|
| | | | | | (95% | Pseudo |
| Division | Coef. | SE | z | p | CI) | R2 |
| NGAL | 0.02812 | 0.04892 | 0.5749 | 0.5654 | −0.07033 | 0.4538 |
| (ng/mL) | | | | | to 0.1187 | |
| KIM-1 | 1.078 | 0.2612 | 4.126 | <0.0001 | 0.6224 | |
| (ng/mL) | | | | | to 1.651 | |
| Age | 0.2033 | 0.08362 | 2.431 | 0.015 | 0.04983 | |
| | | | | | to 0.3815 | |
| Constant | −7.344 | 1.432 | 5.127 | <0.0001 | −10.54 | |
| | | | | | to −4.864 | |

n = 111,
LR chi2(3) = 68.65,
p < .0001

The estimated function equation from the logistic regression analysis of Model 16 (TNKA) is as follows:

$$TNKA ( y ) = 0.02812 \times pNGAL ( ng / ml ) + 1.078 \times pKIM - 1 ( ng / ml ) + 0.2033 \times Age ( year ) - 7.344 \qquad [ Equation 18 ]$$

TABLE 40
Model 17: TNKP

| | Crude | | | | | |
|---|---|---|---|---|---|---|
| | | | | | (95% | Pseudo |
| Division | Coef. | SE | z | p | CI) | R2 |
| NGAL | −0.1125 | 0.09665 | 1.164 | 0.2446 | −0.3175 | 0.5221 |
| (ng/mL) | | | | | to 0.05715 | |
| KIM-1 | 1.42 | 0.3875 | 3.664 | 0.0002 | 0.7649 | |
| (ng/mL) | | | | | to 2.302 | |
| Inorganic | 0.7162 | 0.419 | 1.709 | 0.0874 | −0.03583 | |
| Phosphorus | | | | | to 1.642 | |
| Constant | −8.453 | 2.342 | 3.609 | 0.0003 | −14.03 | |
| | | | | | to −4.623 | |

n = 67,
LR chi2(3) = 48.42,
p < .0001

The estimated function equation from the logistic regression analysis of Model 17 (TNKP) is as follows:

$$TNKP ( y ) = - 0.1125 \times pNGAL ( ng / ml ) + 1.42 \times pKIM - 1 ( ng / ml ) + 0.7162 \times Inorganic phosphorus ( mg / dl ) - 8.453 \qquad [ Equation 19 ]$$

TABLE 41
Model 18: TNKAM

| | Crude | | | | | |
|---|---|---|---|---|---|---|
| | | | | | (95% | Pseudo |
| Division | Coef. | SE | z | p | CI) | R2 |
| NGAL | −0.05275 | 0.08639 | 0.6105 | 0.5415 | −0.2389 | 0.4107 |
| (ng/mL) | | | | | to 0.08974 | |
| KIM-1 | 1.001 | 0.4017 | 2.491 | 0.0127 | 0.3142 | |
| (ng/mL) | | | | | to 1.914 | |
| Amylase | 0.001492 | 0.001407 | 1.061 | 0.2889 | −0.001088 | |
| | | | | | to 0.004564 | |
| Constant | −5.468 | 1.622 | 3.371 | 0.0007 | −9.301 | |
| | | | | | to −2.798 | |

n = 45,
LR chi2(3) = 24.51,
p < .0001

The estimated function equation from the logistic regression analysis of Model 18 (TNKAm) is as follows:

$$TNKAm ( y ) = - 0.05275 \times pNGAL ( ng / ml ) + 1.001 \times pKIM - 1 ( ng / ml ) + 0.001492 \times Amylase ( U / L ) - 5.468 \qquad [ Equation 20 ]$$

TABLE 42
Model 19: TNKCS

| | Crude | | | | | |
|---|---|---|---|---|---|---|
| | | | | | (95% | Pseudo |
| Division | Coef. | SE | z | p | CI) | R2 |
| NGAL | −0.05199 | 0.1035 | 0.5023 | 0.6155 | −0.2740 | 0.8344 |
| (ng/mL) | | | | | to 0.1515 | |
| KIM-1 | 0.2173 | 0.3619 | 0.6005 | 0.5482 | −0.4621 | |
| (ng/mL) | | | | | to 1.074 | |
| SDMA | 0.9584 | 0.3288 | 2.915 | 0.0036 | 0.4763 | |
| | | | | | to 1.828 | |
| Creatinine | 9.823 | 3.506 | 2.802 | 0.0051 | 4.663 | |
| | | | | | to 19.17 | |
| Constant | −26.57 | 9.008 | 2.95 | 0.0032 | −51.09 | |
| | | | | | to −13.96 | |

n = 111,
LR chi2(4) = 126.2,
p < .0001

The estimated function equation from the logistic regression analysis of Model 19 (TNKCS) is as follows:

$$TNKCS ( y ) = - 0.05199 \times pNGAL ( ng / ml ) + 0.2173 \times pKIM - 1 ( ng / ml ) + 9.823 \times sCr ( mg / dl ) + 0.9584 \times SDMA ( μg / dl ) - 26.57 \qquad [ Equation 21 ]$$

TABLE 43
Model 20: TNKB

| | Crude | | | | | |
|---|---|---|---|---|---|---|
| | | | | | (95% | Pseudo |
| Division | Coef. | SE | z | p | CI) | R2 |
| NGAL | −0.0266 | 0.05992 | 0.4439 | 0.6571 | −0.1428 | 0.5609 |
| (ng/mL) | | | | | to 0.08654 | |
| KIM-1 | 1 | 0.2599 | 3.848 | 0.0001 | 0.5480 | |
| (ng/mL) | | | | | to 1.577 | |
| BUN | 0.11 | 0.03181 | 3.459 | 0.0005 | 0.05685 | |
| | | | | | to 0.1829 | |
| Constant | −7.155 | 1.287 | 5.559 | <0.0001 | −10.05 | |
| | | | | | to −4.927 | |

n = 111,
LR chi2(3) = 84.85,
p < .0001

The estimated function equation from the logistic regression analysis of Model 20 (TNKB) is as follows:

$$TNKB ( y ) = - 0.0266 \times pNGAL ( ng / ml ) + 1 \times pKIM - 1 ( ng / ml ) + 0.11 \times BUN ( mg / dl ) - 7.155 \qquad [ Equation 22 ]$$

TABLE 44
Model 21: TNKCSB

| | Crude | | | | | |
|---|---|---|---|---|---|---|
| | | | | | (95% | Pseudo |
| Division | Coef. | SE | z | p | CI) | R2 |
| NGAL | −0.08838 | 0.1169 | 0.7561 | 0.4496 | −0.3505 | 0.8433 |
| (ng/mL) | | | | | to 0.1301 | |
| KIM-1 | 0.2262 | 0.3563 | 0.6349 | 0.5255 | −0.4688 | |
| (ng/mL) | | | | | to 1.054 | |
| SDMA | 0.961 | 0.3357 | 2.863 | 0.0042 | 0.4645 | |
| | | | | | to 1.838 | |
| Creatinine | 8.739 | 3.503 | 2.495 | 0.0126 | 3.468 | |
| | | | | | to 18.02 | |
| BUN | 0.04618 | 0.0436 | 1.059 | 0.2896 | −0.02961 | |
| | | | | | to 0.1558 | |
| Constant | −26.28 | 8.895 | 2.955 | 0.0031 | −50.50 | |
| | | | | | to −13.79 | |

n = 111,
LR chi2(5) = 127.6,
p < .0001

The estimated function equation from the logistic regression analysis of Model 21 (TNKCSB) is as follows:

$$TNKCSB ( y ) = - 0.08838 \times pNGAL ( ng / ml ) + 0.2262 \times pKIM - 1 ( ng / ml ) + 8.739 \times sCr ( mg / dl ) + 0.961 \times SDMA ( μg / dl ) + 0.04618 \times BUN ( mg / dL ) - 26.28 \qquad [ Equation 23 ]$$

Referring to Tables 36 to 44, Model 21 (TNKCSB), which combines creatinine, SDMA and BUN with pNGAL, pKIM-1, and the like was found to be the optimal model (function) with the highest explanatory power (Pseudo R2) of 84.33%.

Subsequently, any one of the values derived from Equations 15 to 23, which are linear equation models, can be transformed to converge to a value of 1 or 0 by using an exponential function. Here, the prediction equation using the exponential function to classify the kidney disease groups (IRIS stages 2 to 4, indicating a value of 1) and the normal group (normal, risk group, and Stage 1 kidney disease, indicating a value of 0) is as shown in Equation 24. In Equation 24, the p value is a percentage with a distribution ranging from 0 to 1, wherein a value of less than 0.5 is determined as having no kidney disease, and a value of 0.5 or more is determined as belonging to the kidney disease groups.

$$ρ = exp ( y ) / [ exp ( y ) + 1 ] \qquad [ Equation 24 ]$$

In the equation above, y is a value derived from Equations 15 to 23.

### Experimental Example 4.2. Cutoff Value Ranges for Diagnosing Stage 2 Kidney Disease

To evaluate the accuracy of the optimal model for diagnosing kidney disease groups derived in Experimental Example 4.1, the ROC curves were analyzed by using the GraphPad Prism program, and the cutoff values for diagnosing kidney disease groups (normal group, risk group, and IRIS stage 1 vs. IRIS stages 2 to 4) for NGAL and KIM-1, and TNK, TNKC, TNKS, TNKA, TNKP, TNKAm, TNKCS, TNKB and TNKCSB models derived in Section 3-1 are shown in Tables 45 to 55 below.

TABLE 45
pNGAL

| Cutoff | Sensitivity | | Specificity | | |
|---|---|---|---|---|---|
| value | (%) | 95% CI | (%) | 95% CI | LR+ |
| >2.086 | 100 | 92.44% to | 21.54 | 13.29% to | 1.275 |
| | | 100.0% | | 32.97% | |
| >4.888 | 82.98 | 69.86% to | 60 | 47.86% to | 2.074 |
| | | 91.11% | | 71.03% | |
| >40.64 | 6.383 | 2.195% to | 100 | 94.42% to | — |
| | | 17.16% | | 100.0% | |

As shown in Table 45, the cutoff value range for the pNGAL concentration to distinguish the kidney disease groups may be from 2.086 ng/ml (sensitivity 100%, specificity 21.54%) to 40.64 ng/ml (sensitivity 6.383%, specificity 100%).

TABLE 46
pKIM-1

| Cutoff | Sensitivity | | Specificity | | |
|---|---|---|---|---|---|
| value | (%) | 95% CI | (%) | 95% CI | LR+ |
| >2.360 | 100 | 92.73% to | 16.42 | 9.423% to | 1.196 |
| | | 100.0% | | 27.06% | |
| >4.137 | 79.59 | 66.36% to | 86.57 | 76.40% to | 5.925 |
| | | 88.52% | | 92.77% | |
| >5.323 | 55.1 | 41.32% to | 100 | 94.58% to | — |
| | | 68.15% | | 100.0% | |

As shown in Table 46, the cutoff value range for the pKIM-1 concentration to distinguish the kidney disease group may be from 2.360 ng/ml (sensitivity 100%, specificity 16.42%) to 5.323 ng/ml (sensitivity 55.1%, specificity 100.0%).

TABLE 47
Model 13: TNK

| Cutoff | Sensitivity | | Specificity | | |
|---|---|---|---|---|---|
| value | (%) | 95% CI | (%) | 95% CI | LR |
| >−5.169 | 100 | 92.44% to | 12.5 | 6.472% to | 1.143 |
| | | 100.0% | | 22.77% | |
| >−4.833 | 78.72 | 65.10% to | 85.94 | 75.38% to | 5.598 |
| | | 88.01% | | 92.42% | |
| >−4.081 | 23.4 | 13.60% to | 100 | 94.34% to | — |
| | | 37.22% | | 100.0% | |

As shown in Table 47, the cutoff value range for the TNK to distinguish the risk group of kidney disease may be from −5.169 (sensitivity 100%, specificity 12.5%) to −4.081 (sensitivity 23.4%, specificity 100.0%).

TABLE 48
Model 14: TNKC

| Cutoff | Sensitivity | | Specificity | | |
|---|---|---|---|---|---|
| value | (%) | 95% CI | (%) | 95% CI | LR |
| >−3.718 | 100 | 92.44% to | 15.63 | 8.715% to | 1.185 |
| | | 100.0% | | 26.43% | |
| >−0.1381 | 85.11 | 72.31% to | 92.19 | 82.98% to | 10.89 |
| | | 92.59% | | 96.62% | |
| >1.308 | 70.21 | 56.02% to | 100 | 94.34% to | — |
| | | 81.35% | | 100.0% | |

As shown in Table 48, the cutoff value range for the TNKC to distinguish the risk group of kidney disease may be from −3.718 (sensitivity 100%, specificity 15.63%) to 1.308 (sensitivity 70.21%, specificity 100.0%).

TABLE 49
Model 15: TNKS

| Cutoff | Sensitivity | | Specificity | | |
|---|---|---|---|---|---|
| value | (%) | 95% CI | (%) | 95% CI | LR |
| >−3.567 | 100 | 92.44% to | 39.06 | 28.06% to | 1.641 |
| | | 100.0% | | 51.31% | |
| >−0.1465 | 87.23 | 74.83% to | 98.44 | 91.67% to | 55.83 |
| | | 94.02% | | 99.92% | |
| >−0.08390 | 87.23 | 74.83% to | 100 | 94.34% to | — |
| | | 94.02% | | 100.0% | |

As shown in Table 49, the cutoff value range for the TNKS to distinguish the risk group of kidney disease may be from −3.567 (sensitivity 100%, specificity 39.06%) to −0.08390 (sensitivity 87.23%, specificity 100.0%).

TABLE 50
Model 16: TNKA

| Cutoff | Sensitivity | | Specificity | | |
|---|---|---|---|---|---|
| value | (%) | 95% CI | (%) | 95% CI | LR |
| >−4.284 | 100 | 92.44% to | 3.125 | 0.5553% to | 1.032 |
| | | 100.0% | | 10.70% | |
| >−0.2974 | 80.85 | 67.46% to | 87.5 | 77.23% to | 6.468 |
| | | 89.58% | | 93.53% | |
| >1.647 | 51.06 | 37.24% to | 100 | 94.34% to | — |
| | | 64.72% | | 100.0% | |

As shown in Table 50, the cutoff value range for the TNKA to distinguish the risk group of kidney disease may be from −4.284 (sensitivity 100%, specificity 3.125%) to 1.647 (sensitivity 51.06%, specificity 100.0%).

TABLE 51
Model 17: TNKP

| Cutoff | Sensitivity | | Specificity | | |
|---|---|---|---|---|---|
| value | (%) | 95% CI | (%) | 95% CI | LR |
| >−1.775 | 100 | 89.28% to | 54.29 | 38.19% to | 2.188 |
| | | 100.0% | | 69.53% | |
| >−0.5240 | 87.5 | 71.93% to | 85.71 | 70.62% to | 6.125 |
| | | 95.03% | | 93.74% | |
| >1.308 | 70.21 | 56.02% to | 100 | 94.34% to | — |
| | | 81.35% | | 100.0% | |

As shown in Table 51, the cutoff value range for the TNKP to distinguish the risk group of kidney disease may be from −1.775 (sensitivity 100%, specificity 54.29%) to 1.308 (sensitivity 70.21%, specificity 100.0%).

TABLE 52
Model 18: TNKAm

| Cutoff | Sensitivity | | Specificity | | |
|---|---|---|---|---|---|
| value | (%) | 95% CI | (%) | 95% CI | LR |
| >−2.121 | 100 | 81.57% to | 50 | 32.63% to | 2 |
| | | 100.0% | | 67.37% | |
| >−0.5133 | 82.35 | 58.97% to | 85.71 | 68.51% to | 5.765 |
| | | 93.81% | | 94.30% | |
| >1.374 | 47.06 | 26.17% to | 100 | 87.94% to | — |
| | | 69.04% | | 100.0% | |

As shown in Table 52, the cutoff value range for the TNKAm to distinguish the risk group of kidney disease may be from −2.121 (sensitivity 100%, specificity 50%) to 1.374 (sensitivity 47.06%, specificity 100.0%).

TABLE 53
Model 19: TNKCS

| Cutoff | Sensitivity | | Specificity | | |
|---|---|---|---|---|---|
| value | (%) | 95% CI | (%) | 95% CI | LR |
| >−0.7587 | 100 | 92.44% to | 92.19 | 82.98% to | 12.8 |
| | | 100.0% | | 96.62% | |
| >−0.6824 | 97.87 | 88.89% to | 92.19 | 82.98% to | 12.53 |
| | | 99.89% | | 96.62% | |
| >1.575 | 80.85 | 67.46% to | 98.44 | 91.67% to | 51.74 |
| | | 89.58% | | 99.92% | |
| >2.234 | 80.85 | 67.46% to | 100 | 94.34% to | — |
| | | 89.58% | | 100.0% | |

As shown in Table 53, the cutoff value range for the TNKCS to distinguish the risk group of kidney disease may be from −0.7587 (sensitivity 100%, specificity 92.19%) to 2.234 (sensitivity 80.85%, specificity 100.0%).

TABLE 54
Model 20: TNKB

| Cutoff | Sensitivity | | Specificity | | |
|---|---|---|---|---|---|
| value | (%) | 95% CI | (%) | 95% CI | LR |
| >−4.175 | 100 | 92.44% to | 4.688 | 1.278% to | 1.049 |
| | | 100.0% | | 12.90% | |
| >0.1259 | 82.98 | 69.86% to | 98.44 | 91.67% to | 53.11 |
| | | 91.11% | | 99.92% | |
| >1.734 | 70.21 | 56.02% to | 100 | 94.34% to | — |
| | | 81.35% | | 100.0% | |

As shown in Table 54, the cutoff value range for the TNKB to distinguish the risk group of kidney disease may be from −4.175 (sensitivity 100%, specificity 4.688%) to 1.734 (sensitivity 70.21%, specificity 100.0%).

TABLE 55
Model 21: TNKCSB

| Cutoff | Sensitivity | | Specificity | | |
|---|---|---|---|---|---|
| value | (%) | 95% CI | (%) | 95% CI | LR |
| >−1.657 | 100 | 92.44% to | 90.63 | 81.02% to | 10.67 |
| | | 100.0% | | 95.63% | |
| >−0.5159 | 97.87 | 88.89% to | 93.75 | 85.00% to | 15.66 |
| | | 99.89% | | 97.54% | |
| >1.506 | 85.11 | 72.31% to | 100 | 94.34% to | — |
| | | 92.59% | | 100.0% | |

As shown in Table 55, the cutoff value range for the TNKCSB to distinguish the risk group of kidney disease may be from −1.657 (sensitivity 100%, specificity 90.63%) to 1.506 (sensitivity 85.11%, specificity 100.0%).

### Experimental Example 4.3. Evaluation of Diagnostic Accuracy of Derived Optimal Model for Stage 2 Kidney Disease

The ROC curves were analyzed by using the GraphPad Prism program, and from the cutoff value ranges for pNGAL and pKIM-1, and indexes TNK, TNKC, TNKS, TNKA, TNKP, TNKAm, TNKCS, TNKB and TNKCSB derived in Section 4-2, the optimal cutoff value with the highest diagnostic accuracy was derived (FIG. 5). In addition, the ranges of diagnostic sensitivity, specificity, accuracy, and reference standard for distinguishing between the normal control, the risk group, and IRIS stage 1 and IRIS stages 2 to 4 are shown in Table 56 below.

TABLE 56

| | | | | | Positive | Negative |
|---|---|---|---|---|---|---|
| | Cut-off | AUC | Sensitivity % | Specificity % | likelihood | likelihood |
| Division | (unit) | (95% CI) | (95% CI) | (95% CI) | ratio | ratio |
| sCr | 1.25 (mg/dL) | 0.89*** | 69.39 | 95.59 | 15.73 | 0.32 |
| | | (0.83-0.96) | (55.47-80.48) | (87.81-98.8) | | |
| SDMA | 16.50 (μg/dL) | 0.95*** | 85.71 | 98.48 | 56.39 | 0.15 |
| | | (0.9-0.99) | (73.33-92.9) | (91.9-99.92) | | |
| pNGAL | 4.89 (ng/mL) | 0.75*** | 82.98 | 60 | 2.07 | 0.28 |
| | | (0.66-0.84) | (69.86-91.11) | (47.86-71.03) | | |
| pKIM-1 | 4.14 (ng/mL) | 0.88*** | 79.59 | 86.57 | 5.93 | 0.24 |
| | | (0.81-0.95) | (66.36-88.52) | (76.4-92.77) | | |
| TNK | >−4.833 | 0.86*** | 78.72 | 85.94 | 5.60 | 0.25 |
| | | (0.79-0.93) | (65.1-88.01) | (75.38-92.42) | | |
| TNKS | >−0.08390 | 0.95*** | 87.23 | 100 | — | 0.13 |
| | | (0.91-1) | (74.83-94.02) | (94.34-100) | | |
| TNKC | >−0.1381 | 0.94*** | 85.11 | 92.19 | 10.90 | 0.16 |
| | | (0.89-0.99) | (72.31-92.59) | (82.98-96.62) | | |
| TNKA | >−0.2974 | 0.90*** | 80.85 | 87.5 | 6.47 | 0.22 |
| | | (0.84-0.96) | (67.46-89.58) | (77.23-93.53) | | |
| TNKP | >−0.5240 | 0.92*** | 87.5 | 85.71 | 6.12 | 0.15 |
| | | (0.86-0.98) | (71.93-95.03) | (70.62-93.74) | | |
| TNKAm | >−0.5133 | 0.89*** | 82.35 | 85.71 | 5.76 | 0.21 |
| | | (0.8-0.99) | (58.97-93.81) | (68.51-94.3) | | |
| TNKCS | >−0.7587 | 0.99*** | 100 | 92.19 | 12.80 | 0.00 |
| | | (0.98-1) | (92.44-100) | (82.98-96.62) | | |
| TNKB | >0.1259 | 0.92*** | 82.98 | 98.44 | 53.19 | 0.17 |
| | | (0.86-0.98) | (69.86-91.11) | (91.67-99.92) | | |
| TNKCSB | >−0.5159 | 0.99*** | 97.87 | 93.75 | 15.66 | 0.02 |
| | | (0.98-1) | (88.89-99.89) | (85-97.54) | | |

Referring to FIG. 5 and Table 45, the diagnostic accuracy (AUC) for IRIS stages 1 to 4 was high in the order of pNGAL<TNK<pKIM-1<sCr<TNKAm<TNKA<TNKP=TNKB<TNKC<SDMA=TNKS<TNKCS=TNKCSB.

Specifically, an AUC of TNK of the present invention was 0.86 and a value therebelow interpreted as having no kidney disease, i.e., a cutoff value, was-4.833. At the cutoff value, the diagnostic sensitivity was 78.72% and the diagnostic specificity was 85.94%. LR+ (positive likelihood ratio) is 5.60, and LR− (negative likelihood ratio) is 0.25.

In addition, an AUC of TNKCS of the present invention was 0.99 and a value therebelow interpreted as having no kidney disease, i.e., a cutoff value, was 0.7587. At the cutoff value, the diagnostic sensitivity was 100% and the diagnostic specificity was 92.19%. LR+ (positive likelihood ratio) is 12.80, and LR− (negative likelihood ratio) is 0.

In addition, an AUC of TNKCSB of the present invention was 0.99 and a value therebelow interpreted as having no kidney disease, i.e., a cutoff value, was −0.5159. At the cutoff value, the diagnostic sensitivity was 97.87% and the diagnostic specificity was 93.75%. LR+ (positive likelihood ratio) is 15.66, and LR− (negative likelihood ratio) is 0.02.

In contrast, the pNGAL had an AUC of 0.75 and a cutoff value of 4.14 ng/mL. At the cutoff value, the diagnostic sensitivity was 82.98% and the diagnostic specificity was 60%. LR+ (positive likelihood ratio) is 2.07, and LR− (negative likelihood ratio) is 0.28.

In addition, the pKIM-1 had an AUC of 0.88 and a cutoff value of 4.14 ng/ml. At the cutoff value, the diagnostic sensitivity was 78.72% and the diagnostic specificity was 85.94%. LR+ (positive likelihood ratio) is 5.93, and LR− (negative likelihood ratio) is 0.24.

The results above indicate that TNKS, TNKCS and TNKCSB of the present invention can distinguish the kidney disease groups with higher accuracy, sensitivity and specificity than sCr and SDMA. That is, the indexes of the present invention are found to enable diagnosis of kidney disease at a level equivalent to or higher than the conventional indexes.

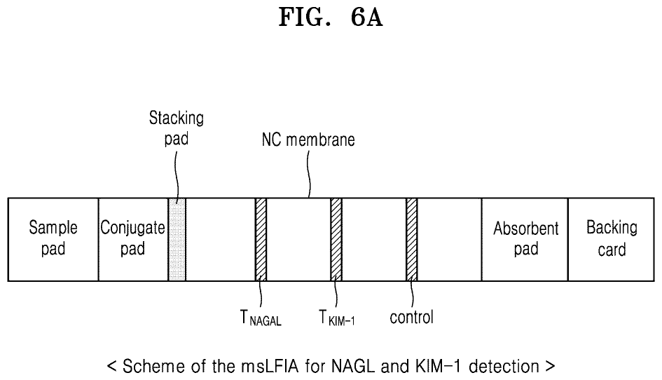

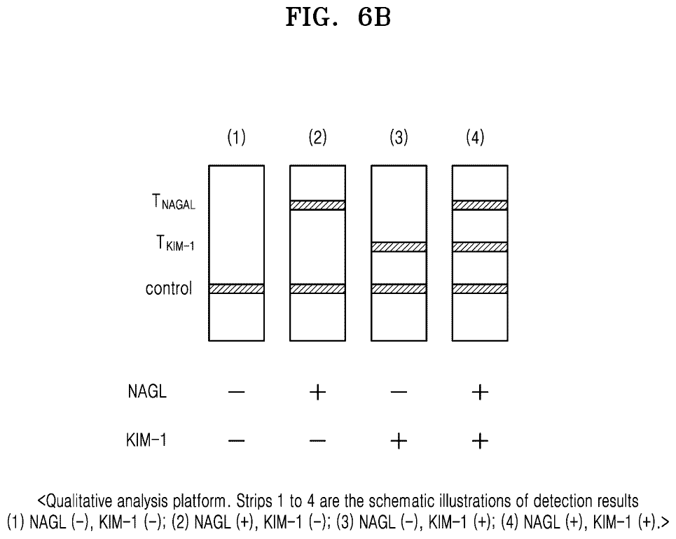

Referring to the results above, a kit for diagnosis of kidney disease was manufactured. The kit is manufactured in a strip structure as shown in FIG. 6A, and includes a sample pad, a conjugate pad, a stacking pad, an NC membrane, an absorbent pad, and a solid support (e.g., a backing card).

As shown in FIG. 6B, depending on the combination of a control line and test lines (KIM-1 and NGAL), the onset of CKD may be determined based on the IRIS guidelines for staging CKD. When determining based on the presence of the control line, the absence of KIM-1 and NGAL can be judged as a normal group, and the presence or absence of KIM-1 and/or NGAL can be judged as a kidney disease group.

Although the present invention has been described with reference to the preferred embodiments mentioned above, various modifications and variations are possible without departing from the spirit and scope of the invention. In addition, the appended claims encompass such modifications or variations that fall within the spirit of the present invention.

1. A composition for diagnosing kidney disease, comprising an agent capable of measuring an expression level of a neutrophil gelatinase-associated lipocalin (NGAL) protein, a kidney injury molecule-1 (KIM-1) protein, or a combination thereof, or a gene encoding the same.

2. The composition of claim 1, wherein the composition is capable of distinguishing between a risk group and each stage based on the International Renal Interest Society (IRIS) guidelines for staging chronic kidney disease (CKD).

3. The composition of claim 1, further comprising an agent capable of measuring an expression level of one or more proteins selected from the group consisting of symmetric dimethylarginine (SDMA), creatinine, inorganic phosphorus, amylase, and BUN, or a gene encoding the same.

4. The composition of claim 1, wherein the agent capable of measuring the expression level of the protein or gene encoding the protein is selected from the group consisting of an antibody, a ligand, a peptide nucleic acid (PNA), an aptamer, and a nanoparticle that bind specifically to the protein, or the group consisting of a primer pair, a probe, and an antisense nucleotide that bind specifically to the gene.

5. The composition of claim 1, wherein the kidney disease is acute kidney injury (AKI) or chronic kidney disease (CKD).

6. The composition of claim 1, wherein the expression level of the protein or gene encoding the same is measured in a body fluid sample of a subject.

7. A kit for diagnosing kidney disease, comprising the composition of claim 1.

8. A method of providing information for diagnosis of kidney disease, comprising: measuring an expression level of a neutrophil gelatinase-associated lipocalin (NGAL) protein, a kidney injury molecule-1 (KIM-1) protein, or a combination thereof, or a gene encoding the same, in a biological sample obtained from a subject; and
comparing the measured expression level with expression levels of proteins of a normal group or a combination thereof, or genes encoding the same.

9. The method of claim 8, further comprising measuring an expression level of one or more proteins selected from the group consisting of symmetric dimethylarginine (SDMA), creatinine, inorganic phosphorus, amylase, and BUN, or a gene encoding the same.

10. The method of claim 8, further comprising setting, as an independent variable, the measured expression level of the protein or gene, and setting, as a dependent variable, an onset of a risk group of kidney disease (a stage with risk factors), Stage 1 chronic kidney disease (CKD) (IRIS stage 1), or Stages 2 to 4 CKD (IRIS stages 2 to 4) based on the International Renal Interest Society (IRIS) guidelines for staging CKD;
modeling a relation between the independent variable and the dependent variable by logistic regression analysis to deduce a model equation; and
determining the subject to belong to the risk group of kidney disease or to be at IRIS stage 1 or IRIS stages 2 to 4, when a value deduced by the model equation is greater than or equal to a predetermined cutoff value.

11. The method of claim 10, wherein the model equation is any one selected from Equations 1 to 6 below:
$$RNK ( y ) = 1.648 \times pNGAL ( ng / ml ) + 3.287 \times pKIM - 1 ( ng / ml ) - 12.2 \qquad [ Equation 1 ]$$
$$RNKC ( y ) = 1.71 \times pNGAL ( ng / ml ) + 3.306 \times pKIM - 1 ( ng / ml ) + 0.9716 \times sCr ( mg / dl ) - 13.22 \qquad [ Equation 2 ]$$
$$RNKS ( y ) = 1.928 \times pNGAL ( ng / mL ) + 3.948 \times pKIM - 1 ( ng / ml ) + 0.4207 \times SDMA ( μg / dl ) - 19.09 \qquad [ Equation 3 ]$$
$$RNKA = 1.398 \times pNGAL ( ng / ml ) + 3.989 \times pKIM - 1 ( ng / ml ) + 0.5979 \times Age ( year ) - 17.02 \qquad [ Equation 4 ]$$
$$RNKR = 2.832 \times pNGAL ( ng / mL ) + 4.726 \times pKIM - 1 ( ng / ml ) + 8.756 \times CRP ( mg / dl ) - 21.36 \qquad [ Equation 5 ]$$
$$RNKCS = 2.15 \times pNGAL ( ng / ml ) + 4.178 \times pKIM - 1 ( ng / ml ) + 1.798 \times sCr ( mg / dl ) + 0.4377 \times SDMA ( μg / dl ) - 21.97 . \qquad [ Equation 6 ]$$

12. The method of claim 11, wherein the cutoff value of the model equation is any number selected from −4.27 to 2.50.

13. The method of claim 10, wherein the model equation is any one selected from Equations 8 to 13 below:
$$SNK ( y ) = 0.5541 \times pNGAL ( ng / ml ) + 0.3766 \times pKIM - 1 ( ng / ml ) - 2.614 \qquad [ Equation 8 ]$$
$$SNKC ( y ) = 0.5522 \times pNGAL ( ng / ml ) + 0.3146 \times pKIM - 1 ( ng / ml ) + 0.4417 \times sCr ( mg / dl ) - 2.792 \qquad [ Equation 9 ]$$
$$SNKS ( y ) = 0.442 \times pNGAL ( ng / ml ) + 0.001992 \times pKIM - 1 ( ng / ml ) + 0.2562 \times SDMA ( μg / dl ) - 4.079 \qquad [ Equation 10 ]$$
$$SNKA ( y ) = 0.447 \times pNGAL ( ng / mL ) + 0.2079 \times pKIM - 1 ( ng / ml ) + 0.2108 \times Age ( year ) - 3.55 \qquad [ Equation 11 ]$$
$$SNKP ( y ) = 0.4599 \times pNGAL ( ng / ml ) + 0.3363 \times pKIM - 1 ( ng / ml ) + 1.004 \times Inorganic phosphorus ( mg / dl ) - 5.678 \qquad [ Equation 12 ]$$
$$SNKCS ( y ) = 0.4406 \times pNGAL ( ng / ml ) + 0.007931 \times pKIM - 1 ( ng / ml ) - 0.07766 \times sCr ( mg / dl ) + 0.258 \times SDMA ( μg / dl ) - 4.048 . \qquad [ Equation 13 ]$$

14. The method of claim 13, wherein the cutoff value of the model equation is any number selected from −1.67 to 4.26.

15. The method of claim 10, wherein the model equation is any one selected from Equations 15 to 23 below:
$$TNK ( y ) = 0.03031 \times pNGAL ( ng / mL ) + 1.187 \times pKIM - 1 ( ng / ml ) - 5.538 \qquad [ Equation 15 ]$$
$$TNKC ( y ) = - 0.01621 \times pNGAL ( ng / ml ) + 0.9737 \times pKIM - 1 ( ng / ml ) + 3.773 \times sCr ( mg / dl ) - 8.309 \qquad [ Equation 16 ]$$
$$TNKS ( y ) = - 0.01627 \times pNGAL ( ng / ml ) + 0.631 \times pKIM - 1 ( ng / ml ) + 0.4914 \times sCr ( mg / dl ) - 10.55 \qquad [ Equation 17 ]$$
$$TNKA ( y ) = 0.02812 \times pNGAL ( ng / ml ) + 1.078 \times pKIM - 1 ( ng / ml ) + 0.2033 \times Age ( year ) - 7.344 \qquad [ Equation 18 ]$$
$$TNKP ( y ) = - 0.1125 \times pNGAL ( ng / mL ) + 1.42 \times pKIM - 1 ( ng / ml ) + 0.7162 \times Inorganic phosphorus ( mg / dl ) - 8.453 \qquad [ Equation 19 ]$$
$$TNKAm ( y ) = - 0.05275 \times pNGAL ( ng / ml ) + 1.001 \times pKIM - 1 ( ng / ml ) + 0.001492 \times Amylase ( U / L ) - 5.468 \qquad [ Equation 20 ]$$
$$TNKCS ( y ) = - 0.05199 \times pNGAL ( ng / ml ) + 0.2173 \times pKIM - 1 ( ng / ml ) + 9.823 \times sCr ( mg / dl ) + 0.9584 \times SDMA ( μg / dl ) - 26.57 \qquad [ Equation 21 ]$$
$$TNKB ( y ) = - 0.0266 \times pNGAL ( ng / ml ) + 1 \times pKIM - 1 ( ng / ml ) + 0.11 \times BUN ( mg / dl ) - 7.155 \qquad [ Equation 22 ]$$
$$TNKCSB ( y ) = - 0.08838 \times pNGAL ( ng / ml ) + 0.2262 \times pKIM - 1 ( ng / ml ) + 8.739 \times sCr ( mg / dl ) + 0.961 \times SDMA ( μg / dl ) + 0.04618 \times BUN ( mg / dL ) - 26.28 . \qquad [ Equation 23 ]$$

16. The method of claim 15, wherein the cutoff value of the model equation is any number selected from −5.17 to 2.23.

17. The method of claim 8, wherein the method is capable of distinguishing between a normal group and a risk or suspected group before IRIS stage 1, with a sensitivity of 90% or greater and a specificity of 95% or greater.
